(19) United States
(12) Patent Application Publication
(10) Pub. No.: US 2023/0051343 A1
(43) Pub. Date: Feb. 16, 2023

(54) AQUEOUS DISPERSIONS OF INSOLUBLE ALPHA-GLUCAN COMPRISING ALPHA-1,3 GLYCOSIDIC LINKAGES

(71) Applicant: NUTRITION & BIOSCIENCE USA 4 INC., Rochester, NY (US)

(72) Inventors: Kyle KIM, Wilmington, DE (US); Hong YANG, Wilmington, DE (US)

(21) Appl. No.: 17/793,080

(22) PCT Filed: Feb. 2, 2021
(86) PCT No.: PCT/US2021/016209

§ 371 Date: Jul. 15, 2022

Related U.S. Application Data
(60) Provisional application No. 62/969,787, filed on Feb. 4, 2020.
(60) Provisional application No. 62/969,784, filed on Feb. 4, 2020.

(51) Int. Cl. A61K 8/73; A61K 47/36; A61Q 19/00; C12P 19/04; C12N 9/10; A23L 33/21

(52) U.S. Cl. CPC A61K 8/73; A61K 47/36; A61Q 19/00; C12P 19/04; C12N 9/1051; A23L 33/21; A61K 2800/654; A61K 2800/412; A23V 2002/00

(57) ABSTRACT

Disclosed herein are methods of producing aqueous dispersions comprising insoluble alpha-glucan having at least 50% alpha-1,3 glycosidic linkages. For example, in addition to dispersing insoluble alpha-glucan that has never been dried, methods are disclosed for effectively dispersing insoluble alpha-glucan that has previously been dried. Further disclosed are aqueous dispersions comprising insoluble alpha-glucan, such as those produced by the disclosed methods. Aqueous dispersions of the present disclosure have enhanced features of viscosity, stability, and particle size distribution, for example. Application of aqueous dispersions in various products and uses are also disclosed.

This application claims the benefit of U.S. Provisional Application Nos. 62/969,787 (filed Feb. 4, 2020) and 62/969,784 (filed Feb. 4, 2020), which are each incorporated herein by reference in their entirety.

### FIELD

The present disclosure is in the field of polysaccharides. For example, the disclosure regards dispersions of insoluble alpha-glucan, methods of their preparation, and various applications of their use.

### BACKGROUND

Driven by a desire to use polysaccharides in various applications, researchers have explored for polysaccharides that are biodegradable and that can be made economically from renewably sourced feedstocks. One such polysaccharide is alpha-1,3-glucan, an insoluble glucan polymer characterized by having alpha-1,3 glycosidic linkages. This polymer has been prepared, for example, using a glucosyltransferase enzyme isolated from Streptococcus salivarius (Simpson et al., Microbiology 141:1451-1460, 1995). Also for example, U.S. Pat. No. 7,000,000 disclosed the preparation of a spun fiber from enzymatically produced alpha-1,3-glucan. Various other glucan materials have also been studied for developing new or enhanced applications. For example, U.S. Patent Appl. Publ. No. 2015/0232819 discloses enzymatic synthesis of several insoluble glucans having mixed alpha-1,3 and -1,6 linkages.

Dispersions of insoluble alpha-1,3-glucan have been described, such as in U.S. Patent Appl. Publ. Nos. 2018/0021238 and 2018/0273731. While there has been some success in preparing aqueous dispersions of alpha-1,3-glucan that has never been dried following its enzymatic synthesis, it has been difficult to adequately disperse dry alpha-1,3-glucan. In particular, it is believed that previous attempts to disperse dry alpha-1,3-glucan have failed to achieve desirable levels of viscosity and stability, for example. Methods and compositions are disclosed herein to address this problem.

### SUMMARY

In one embodiment, the present disclosure concerns a method of producing an aqueous dispersion. Such a method can comprise: (a) providing a first composition comprising at least 88% insoluble alpha-glucan by weight of the first composition, wherein at least 50% of the glycosidic linkages of the insoluble alpha-glucan are alpha-1,3 glycosidic linkages, and (b) mixing at least aqueous liquid and the first composition to produce an aqueous dispersion having about 0.5% to about 10% by weight of the insoluble alpha-glucan, wherein the mixing comprises applying pressure of at least 1000 pounds per square inch (psi) (e.g., at least 7000 psi).

In another embodiment, the present disclosure concerns another method of producing an aqueous dispersion. Such a method can comprise: (a) providing a first composition comprising (i) about 10% to 55% insoluble alpha-glucan by weight of the first composition, and (ii) a balance of water or aqueous solution up to 100% by weight of the first composition, wherein at least 50% of the glycosidic linkages of the insoluble alpha-glucan are alpha-1,3 glycosidic linkages; and (b) mixing at least aqueous liquid and the first composition to produce an aqueous dispersion having about 0.01% to about 8.5% by weight of the insoluble alpha-glucan, wherein the mixing comprises applying pressure of at least 1000 psi.

In another embodiment, the present disclosure concerns an aqueous dispersion produced according to a dispersion method herein.

In another embodiment, the present disclosure concerns an aqueous dispersion comprising about 0.5 wt % to about 10 wt % insoluble alpha-glucan, wherein at least 60% by weight of the insoluble alpha-glucan particles in the aqueous dispersion have a diameter of less than 30 micrometers, the insoluble alpha-glucan is dispersed through at least about 60% of the volume of the aqueous dispersion, and at least 50% of the glycosidic linkages of the insoluble alpha-glucan are alpha-1,3 glycosidic linkages.

In another embodiment, the present disclosure concerns an aqueous dispersion comprising about 0.01 wt % to about 8.5 wt % insoluble alpha-glucan, wherein: at least 90% by weight of the insoluble alpha-glucan particles in the aqueous dispersion have a diameter of less than 30 micrometers, the insoluble alpha-glucan is dispersed through at least about 80% of the volume of the aqueous dispersion, and at least 50% of the glycosidic linkages of the insoluble alpha-glucan are alpha-1,3 glycosidic linkages.

### BRIEF DESCRIPTION OF THE DRAWINGS

FIG. 1: Shown are dispersions of alpha-1,3-glucan after 24 hours of settling. Alpha-1,3-glucan that had never been dried (wet cake, 40 wt % solids), or was dried (95 wt % solids), following its enzymatic synthesis were dispersed in water to 4 wt % solids with either a rotor stator (10000 rpm, 10 minutes) or high pressure homogenizer (8000 psi, three passes). The resulting dispersions were allowed to settle for 24 hours, after which time settling of the dispersed alpha-1,3-glucan was determined. Refer to Example 1.

FIG. 2: Shown are particle size distributions of aqueous dispersions of alpha-1,3-glucan. Alpha-1,3-glucan in both wet cake (40 wt % solids) and dry (95 wt % solids) forms was dispersed to 4 wt % solids under conditions of high shear (8000 psi homogenization) or less shear (10000 rpm with rotor stator), and then analyzed for particle size. Refer to Example 1.

FIG. 3A: Shown are the viscosities (measured under steady state shear of 10 s⁻¹) resulting from using multiple pass dispersal processing of alpha-1,3-glucan with a rotor stator having a stack of two or three 8SF generators. The starting material was 10 wt % insoluble alpha-1,3-glucan. Refer to Example 2.

FIG. 3B: Shown is a comparison of the viscosities (measured under steady state shear of 10 s⁻¹) resulting from using multiple passes with either a colloid mill or rotor stator (having stack of three 8SF generators) to disperse alpha-1,3-glucan. Refer to Example 2.

FIG. 4: Shown are the viscosities (measured under shear of 10 rpm) of high solids alpha-1,3-glucan preparations subjected to dispersion with an HSD for a certain amount of time. The HSD used either 42 or 52 Hz grinding, depending on the HSD blade type used. Viscosity was measured for each dispersion after it was diluted to 8 wt % solids. Refer to Example 3.

### DETAILED DESCRIPTION

The disclosures of all cited patent and non-patent literature are incorporated herein by reference in their entirety.

Unless otherwise disclosed, the terms “a” and “an” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises 100% alpha-glycosidic linkages, or at least about 80%, 81%, 82%, 83%, 84%, 85%, 86%, 87%, 88%, 89%, 90%, 91%, 92%, 93%, 94%, 95%, 96%, 97%, 98%, or 99% alpha-glycosidic linkages. Examples of alpha-glucan polymers herein include alpha-1,3-glucan.

The terms “poly alpha-1,3-glucan”, “alpha-1,3-glucan”, “alpha-1,3-glucan polymer” and the like are used interchangeably herein. Alpha-1,3-glucan is a polymer comprising glucose monomeric units linked together by glycosidic linkages, wherein at least about 50% of the glycosidic linkages are alpha-1,3. Alpha-1,3-glucan in certain embodiments comprises at least 90% or 95% alpha-1,3 glycosidic linkages. Most or all of the other linkages in alpha-1,3-glucan herein typically are alpha-1,6, though some linkages may also be alpha-1,2 and/or alpha-1,4.

The term “copolymer” herein refers to a polymer comprising at least two different types of alpha-glucan, such as dextran and alpha-1,3-glucan. The terms “graft copolymer”, “branched copolymer” and the like herein generally refer to a copolymer comprising a “backbone” (or “main chain”) and side chains branching from the backbone. The side chains are structurally distinct from the backbone. Examples of graft copolymers herein comprise a dextran backbone (or dextran backbone that has been modified with about 1%-35% alpha-1,2 branches, e.g.), and at least one side chain of alpha-1,3-glucan comprising at least about 50% alpha-1,3 glycosidic linkages. An alpha-1,3-glucan side chain herein can have a linkage and molecular weight of alpha-1,3-glucan as disclosed herein, for example. In some aspects, a dextran backbone can have an alpha-1,3-glucan extension, since the non-reducing end(s) of dextran can prime alpha-1,3-glucan synthesis by a glucosyltransferase enzyme.

The terms “dextran”, “dextran polymer”, “dextran molecule” and the like in some aspects herein refer to a water-soluble alpha-glucan comprising all alpha-1,6 glycosidic linkages, or at least about 50%, 60%, 70%, 80%, 90%, 95%, 96%, 97%, 98%, 99%, or 99.5% alpha-1,6 glycosidic linkages (with the balance of the linkages typically being all or mostly alpha-1,3). Enzymes capable of synthesizing dextran from sucrose may be described as “dextransucrases” (EC 2.4.1.5). As used herein, the term “dextranase” (alpha-1,6-glucan-6-glucanohydrolase; EC 3.2.1.11) refers to an enzyme capable of endohydrolysing 1,6-alpha glycosidic linkages.

The terms “glycosidic linkage”, “glycosidic bond”, “linkage” and the like are used interchangeably herein and refer to the covalent bonds connecting the sugar monomers within a saccharide compound (oligosaccharides and/or polysaccharides). The term “alpha-1,3 glycosidic linkage” as used herein refers to the type of covalent bond that joins alpha-D-glucose molecules to each other through carbons 1 and 3 on adjacent alpha-D-glucose rings. The term “alpha-1,6-glycosidic linkage” as used herein refers to the covalent bond that joins alpha-D-glucose molecules to each other through carbons 1 and 6 on adjacent alpha-D-glucose rings. The glycosidic linkages of a glucan polymer herein can also be referred to as “glucosidic linkages”. Herein, “alpha-D-glucose” will be referred to as “glucose”.

The glycosidic linkage profile of an alpha-glucan herein can be determined using any method known in the art. For example, a linkage profile can be determined using methods using nuclear magnetic resonance (NMR) spectroscopy (e.g., ¹³C NMR and/or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, Fla., 2005), which is incorporated herein by reference.

The “molecular weight” of alpha-glucan polymers herein can be represented as weight-average molecular weight (Mw) or number-average molecular weight (Mn), the units of which are in Daltons (Da) or grams/mole. Alternatively, the molecular weight of alpha-glucan polymers can be represented as DPw (weight average degree of polymerization) or DPn (number average degree of polymerization). The molecular weight of smaller alpha-glucan polymers such as oligosaccharides can optionally be provided as “DP” (degree of polymerization), which simply refers to the number of glucoses comprised within the alpha-glucan; “DP” can also characterize the molecular weight of a polymer on an individual molecule basis. Various means are known in the art for calculating these various molecular weight measurements such as with high-pressure liquid chromatography (HPLC), size exclusion chromatography (SEC), or gel permeation chromatography (GPC).

As used herein, Mw can be calculated as Mw=ΣNiMi²/ΣNiMi, where Mi is the molecular weight of an individual chain i and Ni is the number of chains of that molecular weight. Besides SEC, the Mw of a polymer can be determined by other techniques such as static light scattering, mass spectrometry, MALDI-TOF (matrix-assisted laser desorption/ionization time-of-flight), small angle X-ray or neutron scattering, or ultracentrifugation. As used herein, Mn can be calculated as Mn=ΣNiMi/ΣNi where Mi is the molecular weight of a chain i and Ni is the number of chains of that molecular weight. Besides SEC, the Mn of a polymer can be determined by various colligative property methods such as vapor pressure osmometry, end-group determination by spectroscopic methods such as proton NMR, proton FTIR, or UV-Vis. As used herein, DPn and DPw can be calculated from Mw and Mn, respectively, by dividing them by molar mass of the one monomer unit M₁. In the case of unsubstituted glucan polymer, M₁=162. In the case of a substituted (derivatized) glucan polymer, M₁=162+M_{f}×DoS, where M_{f} is molar mass of the substituting group, and DoS is degree of substitution (average number of substituted groups per one glucose unit of the glucan polymer).

The terms “particle”, “particulate” and like terms are interchangeably used herein, and refers to the smallest identifiable unit in a particulate system. A particle of insoluble alpha-glucan in some aspects has an average size of less than about 100 micron (micrometer). The term “particulated” and like terms can be used to characterize particles of insoluble alpha-glucan herein; particulated insoluble alpha-glucan in typical aspects of the present disclosure is as this material exists when dispersed under aqueous conditions. Particle size in some aspects can refer to particle diameter and/or the length of the longest particle dimension. The average size can be based on the average of diameters and/or longest particle dimensions of at least 50, 100, 500, 1000, 2500, 5000, or 10000 or more particles, for example.

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37:D233-238, 2009).

The term “glucosyltransferase catalytic domain” herein refers to the domain of a glucosyltransferase enzyme that provides alpha-glucan-synthesizing activity to a glucosyltransferase enzyme. A glucosyltransferase catalytic domain typically does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components. Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher (e.g., DP100 and higher). It would be understood that certain glucan products, such as alpha-1,3-glucan with a degree of polymerization (DP) of at least 8 or 9, are water-insoluble and thus not dissolved in a glucan synthesis reaction, but rather may be present out of solution (e.g., by virtue of having precipitated from the reaction). It is in a glucan synthesis reaction where the step of contacting water, sucrose and a glucosyltransferase enzyme is performed. The term “under suitable reaction conditions” as used herein refers to reaction conditions that support conversion of sucrose to alpha-glucan product(s) and fructose via glucosyltransferase enzyme activity. It is during such a reaction that glucosyl groups originally derived from the input sucrose are enzymatically transferred and used in alpha-glucan polymer synthesis; glucosyl groups as involved in this process can thus optionally be referred to as the glucosyl component or moiety (or like terms) of a glucosyltransferase reaction.

The “yield” of insoluble alpha-glucan product in a glucosyltransferase reaction in some aspects herein represents the molar yield based on the converted sucrose. The molar yield of an alpha-glucan product can be calculated based on the moles of insoluble alpha-glucan product divided by the moles of the sucrose converted. Moles of converted sucrose can be calculated as follows: (mass of initial sucrose−mass of final sucrose)/molecular weight of sucrose [342 g/mol]. This molar yield calculation can be considered as a measure of selectivity of the reaction toward the insoluble alpha-glucan. In some aspects, the “yield” of insoluble alpha-glucan product in a glucosyltransferase reaction can be based on the glucosyl component of the reaction. Such a yield (yield based on glucosyl) can be measured using the following formula:

Insoluble Alpha-Glucan Yield=((IS/2−(FS/2+LE/2+GL+SO))/(IS/2−FS/2))×100%.

The fructose balance of a glucosyltransferase reaction can be measured to ensure that HPLC data, if applicable, are not out of range (90-110% is considered acceptable). Fructose balance can be measured using the following formula:

Fructose Balance=((180/342×(FS+LE)+FR)/(180/342×IS))×100%.

In the above two formulae, IS is [Initial Sucrose], FS is [Final Sucrose], LE is [Leucrose], GL is [Glucose], SO is [Soluble Oligomers] (gluco-oligosaccharides), and FR is [Fructose]; the concentrations of each foregoing substrate/product provided in double brackets are in units of grams/L and as measured by HPLC, for example.

A “cake” of insoluble alpha-glucan herein refers to a preparation in condensed, compacted, packed, squeezed, and/or compressed form that comprises at least (i) about 45%-90% by weight water or an aqueous solution, and (ii) about 10%-55% by weight insoluble alpha-glucan. A cake in some aspects can be referred to as a “filter cake” or a “wet cake”. A cake herein typically has a soft, solid-like consistency.

The terms “soluble sugars”, “dissolved sugars”, “sugars” and the like herein refer to aqueous-soluble monosaccharides, disaccharides, and/or oligosaccharides. For example, soluble sugars can include at least fructose and/or glucose. As another example, soluble sugars can include at least fructose, glucose, sucrose, leucrose, and/or gluco-oligosaccharides (e.g., DP2-DP7) (e.g., refer to soluble sugars as disclosed in Table 2 of U.S. Patent Appl. Publ. No. 2018/0340199, which is incorporated herein by reference). An example of gluco-oligosaccharides of soluble sugars in some aspects can be as disclosed in Table 17 of U.S. Patent Appl. Publ. No. 2018/0340199 (incorporated above).

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/v %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The terms “dry weight basis” (dwb), “dry solids basis” (dsb) and the like are used interchangeably herein. The amount of a material (e.g., sugars such as fructose, glucose, sucrose, soluble DP2-7 oligosaccharides; optionally salts and impurities) on a dry weight basis in a composition, for example, refers to the weight percentage of that material in the dry/liquid-free portion of the composition.

The term “substantially equivalent” and like terms herein refer to being within (±) 2%, 3%, 5%, or 10% of a given value, for instance. An example of a value herein can be the mixing force(s) (e.g., total mixing forces) applied to a composition comprising aqueous liquid and insoluble alpha-glucan when being dispersed according to a method of the present disclosure.

As used herein, “psi” (pounds per square inch) refers to a unit of pressure. Atmospheric pressure is about 14.7 psi, for example.

The terms “aqueous liquid”, “aqueous fluid”, “aqueous conditions”, “aqueous reaction conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., colloidal dispersions), suspensions and emulsions, for example.

As used herein, the term “colloidal dispersion” refers to a heterogeneous system having a dispersed phase and a dispersion medium, i.e., microscopically dispersed insoluble particles are suspended throughout another substance (e.g., an aqueous composition such as water or aqueous solution). An example of a colloidal dispersion herein is a hydrocolloid. All, or a portion of, the particles of a colloidal dispersion such as a hydrocolloid can comprise insoluble alpha-glucan as presently disclosed. The terms “dispersant” and “dispersion agent” are used interchangeably herein to refer to a material that promotes the formation and/or stabilization of a dispersion. “Dispersing” herein refers to the act of preparing a dispersion of a material in an aqueous liquid. As used herein, the term “latex” (and like terms) refers to a dispersion of one or more types of polymer particles in water or aqueous solution; typically, at least insoluble alpha-glucan particles are in a latex composition as a dispersed polymer component. In some aspects, a latex is an emulsion that comprises a dispersion of at least insoluble alpha-glucan particles.

An alpha-glucan that is “insoluble”, “aqueous-insoluble”, “water-insoluble” (and like terms) (e.g., alpha-1,3-glucan with a DP of 8 or higher) herein does not dissolve (or does not appreciably dissolve) in water or other aqueous conditions, optionally where the aqueous conditions are further characterized to have a pH of 4-9 (e.g., pH 6-8) and/or temperature of about 1 to 85° C. (e.g., 20-25° C.). In contrast, glucans such as certain oligosaccharides herein that are “soluble”, “aqueous-soluble”, “water-soluble” and the like (e.g., alpha-1,3-glucan with a DP less than 8) appreciably dissolve under these conditions.

The term “viscosity” as used herein refers to the measure of the extent to which a fluid (aqueous or non-aqueous) resists a force tending to cause it to flow. Various units of viscosity that can be used herein include centipoise (cP, cps) and Pascal-second (Pas), for example. A centipoise is one one-hundredth of a poise; one poise is equal to 0.100 kg·m⁻¹·s⁻¹.

The term “agitated air drying” and like terms herein refer to a drying process that comprises air drying alpha-glucan while subjecting the alpha-glucan to some sort of movement.

The terms “pressure homogenizer”, “high-pressure homogenizer”, and the like as used herein refer to a device/machine that forces a stream of an aqueous mixture through a system under high pressure (e.g., about 7000 psi), thereby subjecting the mixture to any of a number of forces (e.g., shear forces, impaction, and/or cavitation) that reduce the particle size of the solid material of the mixture.

The term “household care product” and like terms typically refer to products, goods and services relating to the treatment, cleaning, caring and/or conditioning of a home and its contents. The foregoing include, for example, chemicals, compositions, products, or combinations thereof having application in such care.

The terms “fabric”, “textile”, “cloth” and the like are used interchangeably herein to refer to a woven material having a network of natural and/or artificial fibers. Such fibers can be in the form of thread or yarn, for example.

A “fabric care composition” and like terms refer to any composition suitable for treating fabric in some manner. Examples of such a composition include laundry detergents and fabric softeners, which are examples of laundry care compositions.

A “detergent composition” herein typically comprises at least a surfactant (detergent compound) and/or a builder. A “surfactant” herein refers to a substance that tends to reduce the surface tension of a liquid in which the substance is dissolved. A surfactant may act as a detergent, wetting agent, emulsifier, foaming agent, and/or dispersant, for example.

The term “personal care product” and like terms typically refer to products, goods and services relating to the treatment, cleaning, cleansing, caring or conditioning of a person. The foregoing include, for example, chemicals, compositions, products, or combinations thereof having application in such care.

The terms “ingestible product”, “ingestible composition” and the like refer to any substance that, either alone or together with another substance, may be taken orally (i.e., by mouth), whether intended for consumption or not. Thus, an ingestible product includes food/beverage products. “Food/beverage products” refer to any edible product intended for consumption (e.g., for nutritional purposes) by humans or animals, including solids, semi-solids, or liquids. A “food” herein can optionally be referred to as a “foodstuff”, “food product”, or other like term, for example. “Non-edible products” (“non-edible compositions”) refer to any composition that can be taken by the mouth for purposes other than food or beverage consumption. Examples of non-edible products herein include supplements, nutraceuticals, functional food products, pharmaceutical products, oral care products (e.g., dentifrices, mouthwashes), and cosmetic products such as sweetened lip balms. A “pharmaceutical product”, “medicine”, “medication”, “drug” or like term herein refers to a composition used to treat disease or injury, and can be administered enterally or parenterally.

The term “paint” (and like terms) herein is a type of coating composition that is a dispersion of a pigment in a suitable liquid (e.g., aqueous liquid) that can be used to form an adherent coating when spread on a surface in a thin coat. Paint as applied to a surface can provide coloration/decoration and/or protection to the surface. A paint herein, by virtue of further comprising dispersed insoluble alpha-glucan (i.e., a dispersed polymer), can optionally be characterized as a latex or latex paint.

The terms “sequence identity”, “identity” and the like as used herein with respect to a polypeptide amino acid sequence (e.g., that of a glucosyltransferase) are as defined and determined in U.S. Patent Appl. Publ. No. 2017/0002336, which is incorporated herein by reference.

The term “isolated” means a substance (or process) in a form or environment that does not occur in nature. A non-limiting example of an isolated substance includes any non-naturally occurring substance such as some forms of insoluble alpha-glucan herein and dispersions thereof (as well as the enzymatic reactions and other processes used to prepare it). It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein. These terms can be used to characterize the “over-expression” or “up-regulation” of a polynucleotide encoding a protein, for example.

It is believed that previous attempts to disperse dry alpha-1,3-glucan have failed to achieve desirable levels of viscosity and stability. Methods and compositions are disclosed herein to address this problem.

Some embodiments of the present disclosure concern a method of producing an aqueous dispersion. Such a method can comprise: (a) providing a first composition comprising at least about 88% insoluble alpha-glucan by weight of the first composition, wherein at least about 50% of the glycosidic linkages of the insoluble alpha-glucan are alpha-1,3 glycosidic linkages, and (b) mixing at least aqueous liquid and the first composition to produce an aqueous dispersion having about 0.5% to about 10% by weight of the insoluble alpha-glucan, wherein the mixing comprises (i) applying pressure of at least about 1000 pounds per square inch (psi) (e.g., at least 7000 psi), or (ii) applying forces that are substantially equivalent to those forces that are applied in (i). This method, which can optionally be characterized as a dispersion or mixing method, is advantageous: while previous attempts to disperse dry alpha-1,3-glucan into an aqueous liquid have failed to achieve desirable levels of viscosity and stability, the present method provides both these features.

A dispersion method in some aspects can comprise a step of providing a first composition comprising at least 88% insoluble alpha-glucan by weight of the first composition. The first composition is then dispersed/mixed into an aqueous liquid to form an aqueous dispersion. A first composition can have, for example, about, or at least about, 88%, 89%, 90%, 91%, 92%, 93%, 94%, 95%, 96%, 97%, 98%, 99%, 99.5%, 99.9%, or 99.99% insoluble alpha-glucan by weight of the first composition. The balance of the first composition can be an aqueous liquid/fluid such as water or an aqueous solution. Thus, a first composition can optionally be characterized as being dry or dried. A first composition can be in the form of powder, granules, microcapsules, flakes, or any other form of particulate matter, for example. In some alternative aspects, however, a dispersion method can comprise a step of providing a first composition comprising (i) about 10% to 55% insoluble alpha-glucan by weight of the first composition (e.g., any wt % as disclosed for a wet cake of insoluble alpha-glucan disclosed herein), and (ii) a balance of water or aqueous solution up to 100% by weight of the first composition. A first composition in such alternative aspects can comprise any wt % of insoluble alpha-glucan as disclosed for a wet cake herein, for example.

Typically, at least about 50% of the glycosidic linkages of insoluble alpha-glucan used in a dispersion method herein are alpha-1,3 glycosidic linkages. Insoluble alpha-glucan in some aspects can comprise about, or at least about, 40%, 50%, 60%, 70%, 80%, 85%, 90%, 91%, 92%, 93%, 94%, 95%, 96%, 97%, 98%, 99%, 99.5%, or 100% alpha-1,3 glycosidic linkages. In some aspects, accordingly, insoluble alpha-glucan has less than about 60%, 50%, 40%, 30%, 20%, 15%, 10%, 9%, 8%, 7%, 6%, 5%, 4%, 3%, 2%, 1%, 0.5%, or 0% glycosidic linkages that are not alpha-1,3. In general, the glycosidic linkages that are not alpha-1,3 are mostly or entirely alpha-1,6. In certain embodiments, insoluble alpha-glucan has no branch points or less than about 5%, 4%, 3%, 2%, or 1% branch points as a percent of the glycosidic linkages in the glucan. In aspects in which alpha-glucan comprises 50% alpha-1,3 glycosidic linkages, such glucan does not comprise alternan (alternating alpha-1,3 and -1,6 linkages).

In some aspects, the DPw, DPn, or DP of insoluble alpha-glucan is at least about 100. The DPw, DPn, or DP of insoluble alpha-glucan in some aspects can be about, at least about, or less than about, 15, 20, 25, 30, 35, 40, 45, 50, 55, 60, 65, 70, 75, 80, 85, 90, 95, 100, 150, 200, 300, 400, 500, 600, 700, 800, 900, 1000, 1100, 1200, 1300, 1400, 1500, 1600, or 1650. DPw, DPn, or DP can optionally be expressed as a range between any two of these values. Merely as examples, the DPw, DPn, or DP of insoluble alpha-glucan herein can be about 100-1650, 200-1650, 300-1650, 400-1650, 500-1650, 600-1650, 700-1650, 100-1250, 200-1250, 300-1250, 400-1250, 500-1250, 600-1250, 700-1250, 100-1000, 200-1000, 300-1000, 400-1000, 500-1000, 600-1000, 700-1000, 100-900, 200-900, 300-900, 400-900, 500-900, 600-900, 700-900, 15-100, 25-100, 35-100, 15-80, 25-80, 35-80, 15-60, 25-60, 35-60, 15-55, 25-55, 25-50, 35-55, 35-50, 35-45, 35-40, 40-100, 40-80, 40-60, 40-55, 40-50, 45-60, 45-55, or 45-50. DP can be referenced, for example, for alpha-glucan of relatively low molecular weight such as below 200, 150, or 100.

Insoluble alpha-glucan of a dispersion method herein is typically produced by an enzymatic reaction comprising at least water, sucrose and a glucosyltransferase enzyme that synthesizes the insoluble alpha-glucan. Glucosyltransferases, reaction conditions, and/or processes contemplated to be useful for producing insoluble alpha-glucan herein are disclosed in U.S. Pat. Nos. 7,000,000, 8,871,474, 10,301,604 and 10,260,053, U.S. Patent Appl. Publ. Nos. 2019/0112456, 2019/0078062, 2019/0078063, 2018/0340199, 2018/0021238, 2018/0273731, 2017/0002335 and 2015/0064748, and Int. Patent Appl. Publ. No. WO2017/079595, for example, all of which are incorporated herein by reference.

In some aspects, a glucosyltransferase enzyme for producing an insoluble alpha-glucan can comprise an amino acid sequence that is 100% identical to, or at least 90%, 91%, 92%, 93%, 94%, 95%, 96%, 97%, 98%, 98.5%, 99%, or 99.5% identical to, SEQ ID NO:2, 4, 6, 8, 10, 12, 14, 16, 18, 20, 26, 28, 30, 34, or 59, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20, and have glucosyltransferase activity; these amino acid sequences are disclosed in U.S. Patent Appl. Publ. No. 2019/0078063, which is incorporated herein by reference. It is noted that a glucosyltransferase enzyme comprising SEQ ID NO:2, 4, 8, 10, 14, 20, 26, 28, 30, 34, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO:20, can synthesize insoluble alpha-glucan comprising at least about 90% (˜100%) alpha-1,3 linkages. Any of the foregoing glucosyltransferase enzyme amino acid sequences can be modified as described below to increase product yield.

A glucosyltransferase enzyme for producing an insoluble alpha-glucan can, in some aspects, synthesize insoluble alpha-glucan at a yield of at least about 40%. The yield of insoluble alpha-glucan by a glucosyltransferase enzyme in some aspects can be about, or at least about, 40%, 45%, 50%, 55%, 60%, 65%, 70%, 75%, 80%, 85%, 90%, 91%, 92%, 93%, 94%, 95%, or 96%. Yield in some aspects can be measured based on the glucosyl component of the reaction. Yield in some aspects can be measured using HPLC or NIR spectroscopy. Yield can be achieved in a reaction conducted for about 16-24 hours (e.g., ˜20 hours), for example. Examples of such a glucosyltransferase enzyme are those having an amino acid sequence modified such that the enzyme produces more products (insoluble alpha-glucan and fructose), and less by-products (e.g., glucose, oligosaccharides such as leucrose), from a given amount of sucrose substrate. For example, one, two, three, four, or more amino acid residues of the catalytic domain of a glucosyltransferase herein can be modified/substituted to obtain an enzyme that produces more products. Examples of a suitable modified glucosyltransferase enzyme are disclosed in Tables 3-7 of U.S. Patent Appl. Publ. No. 2019/0078063. A modified glucosyltransferase enzyme, for example, can comprise one or more amino acid substitutions corresponding with those in Tables 3-7 (ibid.) that is/are associated with an insoluble alpha-glucan yield of at least 40% (the position numbering of such at least one substitution corresponds with the position numbering of SEQ ID NO:62 as disclosed in U.S. Patent Appl. Publ. No. 2019/0078063). A set of amino acid modifications as listed in Tables 6 or 7 (ibid.) can be used, for example. Thus, in some aspects, insoluble alpha-glucan provided in the first composition of a dispersion method herein was produced in an enzymatic reaction comprising at least water, sucrose and a glucosyltransferase enzyme that synthesizes insoluble alpha-glucan at a yield of at least about 75% (or any other yield as listed above).

The temperature of an enzymatic reaction for producing an insoluble alpha-glucan can be controlled, if desired, and can be about 5-50° C., 20-40° C., 30-40° C., 20-30° C., 20-25° C., 20° C., 25° C., 30° C., 35° C., or 40° C., for example. An enzymatic reaction can be conducted for about, at least about, or up to about, 1, 1.5, 2, 2.5, 3, 3.5, 4, 6, 8, 10, 12, 14, 16, 18, 20, 22, 24, 36, 48, 60, 72, 96, 120, 144, 168, 1-4, 1-3.5, 1-3, 1.5-4, 1.5-3.5, 1.5-3, 2-4, 2-3.5, or 2-3 hours, for example. The pH of an enzymatic reaction in some aspects can be about 4.0, 4.5, 5.0, 5.5, 6.0, 6.5, 7.0, 7.5, 8.0, 4.0-9.0, 4.0-8.5, 4.0-8.0, 5.0-8.0, 5.5-7.5, or 5.5-6.5.

The initial concentration of sucrose in an enzymatic reaction for producing an insoluble alpha-glucan can be about, at least about, or less than about, 10, 15, 20, 25, 30, 40, 45, 50, 55, 60, 80, 90, 95, 100, 105, 110, 125, 150, 200, 300, 400, 500, 600, 10-50, 10-40, 10-30, 10-25, 15-50, 15-40, 15-30, or 15-25 g/L, or a range between any two of these values. “Initial concentration of sucrose” refers to the sucrose concentration in a reaction composition just after all the reaction components have been added/combined (e.g., at least water, sucrose, glucosyltransferase enzyme).

In some aspects, an enzymatic reaction for producing an insoluble alpha-glucan can further comprise soluble gluco-oligosaccharide byproducts from a previously performed enzymatic reaction that produced insoluble alpha-glucan with at least 50% alpha-1,3-linkages. For example, soluble fraction (e.g., filtrate, precipitate) obtained from an enzymatic reaction that produced insoluble alpha-glucan with at least 50% (e.g., 95 or 99%) alpha-1,3-linkages can be added to an enzymatic reaction herein for producing an insoluble alpha-glucan; such soluble fraction contains soluble gluco-oligosaccharide byproducts. Various ways to apply this approach herein are disclosed in U.S. Patent Appl. Publ. No. 2018/0340199, which is incorporated herein by reference. Thus, in some aspects, insoluble alpha-glucan provided in the first composition of a dispersion method herein was produced in an enzymatic reaction comprising at least water, sucrose, a glucosyltransferase enzyme that synthesizes insoluble alpha-glucan, and oligosaccharides that (i) comprise alpha-1,3 and alpha-1,6 glycosidic linkages, and/or (ii) are produced from a glucosyltransferase reaction, wherein the oligosaccharides were added during preparation of the enzymatic reaction.

Insoluble alpha-glucan used for preparing an aqueous dispersion herein typically is enzymatically derived in an inert vessel (typically under cell-free conditions), and is not derived from a cell wall (e.g., fungal cell wall).

Insoluble alpha-glucan typically is processed after its enzymatic production (above) to prepare a first composition for a dispersion method herein. In some aspects, such processing of insoluble alpha-glucan can include at least conducting a step of centrifugation, filtration, fractionation, chromatographic separation, dialysis, evaporation, or dilution. Processing of insoluble alpha-glucan can include at least conducting a step of preparing a cake (wet cake) of insoluble alpha-glucan; insoluble alpha-glucan prepared in this or a related manner can optionally be characterized as being wet insoluble alpha-glucan, or never-dried insoluble alpha-glucan, since it has not been dried since its enzymatic synthesis. Cake preparation can include at least conducting a step of centrifugation (cake is pelleted alpha-glucan) and/or filtration (cake is filtered alpha-glucan), for example. For instance, cake herein can be obtained using a funnel, filter (e.g., a surface filter such as a rotary vacuum-drum filter, cross-flow filter, screen filter, belt filter, screw press, or filter press with or with membrane squeeze capability; or a depth filter such as a sand filter), and/or centrifuge; filtration can be by gravity, vacuum, or press filtration, for instance. Processing can optionally further comprise washing the centrifuged and/or filtered insoluble alpha-glucan one, two, or more times with water or other aqueous liquid. A wash volume can optionally be at least about 10-100% of the volume of the reaction composition used to produce the insoluble alpha-glucan. Washing can be done by various modes, as desired, such as by displacement or re-slurry washing. In some aspects, the aqueous portion of the resulting cake, has no (detectable) dissolved sugars (soluble sugars), or about, or less than about, 0.1-1.5, 0.1-1.25, 0.1-1.0, 0.1-0.75, 0.1-0.5, 0.2-0.6, 0.3-0.5, 0.3-0.4, 0.2, 0.3, 0.4, 0.5, or 0.6 wt % dissolved sugars (in some aspects, any of these wt % values can be with respect to the total weight of the cake itself, or with respect to the cake on a dry weight basis). Such dissolved sugars can include sucrose, fructose, glucose, leucrose, and/or soluble gluco-oligosaccharides, for example. The aqueous portion in a cake in some aspects can have one or more salts/buffers (e.g., Na⁺, Cl⁻, NaCl, phosphate, tris, citrate) (e.g., 0.1, 0.5, or 1.0 wt %) and/or a pH as listed above for glucosyltransferase reaction conditions (e.g., pH 6.0-8.0). In some aspects, the solvent of the aqueous portion herein can comprise about, or at least about, 80, 85, 90, 95, 96, 97, 98, 99, 99.5, or 100 wt % water; the rest of the solvent can be a polar organic solvent, for example. A cake of insoluble alpha-glucan herein can comprise, for example, (i) about 45%-90% or 50%-90% by weight aqueous fluid (e.g., water or aqueous solution), and (ii) about 10%-55% or 10%-50% by weight insoluble alpha-glucan. A cake in some aspects can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 10-55, 10-50, 10-40, 10-30, 10-20, 20-55, 20-50, 20-40, 20-30, 30-55, 30-50, 30-40, 40-55, 40-50, 30-45, 35-45, 37.5-42.5, 35-40, or 40-45 wt % insoluble alpha-glucan, for example (with aqueous fluid adding up to 100 wt %). In some aspects, the insoluble alpha-glucan was in the form of a wet cake prior to being provided in the first composition of a dispersion method herein, wherein the wet cake comprised any of the above amounts of insoluble alpha-glucan (e.g., about 10-55 wt %) and aqueous fluid (e.g., about 45-90 wt %).

Wet insoluble alpha-glucan (e.g., wet cake) can optionally be broken down into smaller particles; such can optionally be done before drying it in some aspects to form a first composition. Providing particles in some aspects can comprise contacting wet insoluble alpha-glucan with a suitable particle-forming device, such that particles of the first composition with an average size of about 0.1-10 mm, for example, are produced. Particle formation herein can optionally be characterized as granulation or particulation. A particulation step in some aspects can be performed such that particles of a certain average size range are directly prepared; this can be done using an suitable particle-forming device with appropriate size dimensions, for example. Additionally or alternatively, particles of a certain average size range can be prepared by applying a suitable size selection tool (e.g., screen/sieve) to a population of particles. Particulation can optionally be performed using an wet insoluble alpha-glucan that has first been chopped, crumbled, and/or otherwise broken into pieces smaller than the original form. A particulation device in some aspects can be a shredder, shaver, grater, tumbler, screen, sieve, grinder, or mill, for example. One or more particulation devices can be employed, as desired. A particulation device in some aspects comprises a plurality of 0.1-mm to 10-mm passages through which wet insoluble alpha-glucan is forcibly transited. Examples of such a device include a screen or sieve (such as that of a grater). The dimensions of the passages (e.g., mesh size) of a screen/sieve herein can be any of those dimensions listed above for average particle sizes, for example. For example, a screen/sieve can have approximately 1-, 2-, 3-, 4-, 5-, 6-, 7-, 8-, 9-, and/or 10-mm passages. Also for example, a screen/sieve can have mesh with the following approximate passage sizes (corresponding to certain commercially available screens/sieves): 9.5, 8.0, 6.7, 6.4, 6.3, 5.7, 5.7, 4.8, 4.0, 3.4, 2.8, 2.4, 2.0, 1.7, 1.4, 1.2, 1.0, 0.8, 0.7, 0.6, 0.5, 0.4, 0.35, 0.3, 0.25, 0.2, 0.15, or 0.1 mm. Also for example, a screen/sieve can have mesh with the following U.S. sieve/mesh designations: ⅜ in., 5/16 in., 0.265 in., ¼ in., No. 3½, No. 4, No. 5, No. 6, No. 7, No. 8, No. 10, No. 12, No. 14, No. 16, No. 18, No. 20, No. 25, No. 30, No. 35, No. 40, No. 45, No. 50, No. 60, No. 70, No. 80, No. 100, or No. 120. While the passages of a screen/sieve herein typically are square or of another four-cornered shape, the passages can be other shapes (e.g., circular/elliptical) in some aspects. In some aspects, wet insoluble alpha-glucan can be processed with at least one device that mixes/blends/stirs/agitates solid/solid-like/non-liquid materials such as an extruder (e.g., paddle extruder; screw extruder such as a single screw or twin screw extruder; co-rotating or counter-rotating extruder), injection molder, compounder, or kneader. This solid materials processing can be done one, two, three, or more times with a given amount of wet insoluble alpha-glucan, and optionally can be done before or after particulation.

A dispersion method in some aspects comprises drying wet insoluble alpha-glucan to provide a first composition for entry to a dispersion process. Insoluble alpha-glucan as provided in a dry/dried form can comprise about, or no more than about, 12, 10, 8, 6, 5, 4, 3, 2, 1.5, 1.0, 0.5, 0.25, 0.10, 0.05, or 0.01 wt % aqueous fluid, for example. As such, a first composition can have, for example, about, or at least about, 88%, 89%, 90%, 91%, 92%, 93%, 94%, 95%, 96%, 97%, 98%, 98.5%, 99%, 99.5%, 99.75%, 99.9%, 99.95%, or 99.99% insoluble alpha-glucan by weight of the first composition. Drying can be done using an oven, freeze drying, spray drying, and/or by agitated air drying (e.g., agitated filter/film drying such as that under vacuum, fluidized bed drying, rotary drying such as drum drying). Drying in some aspects can be at a temperature of about, or at least about, 20, 25, 30, 40, 50, 60, 70, 80, 90, 100, 110, 120, 130, 140, 20-140, 20-130, 30-50, 35-45, 90-110, or 95-105° C., for example. Typically, insoluble alpha-glucan that has been dried is ground up, or otherwise comminuted, into powder or other particulate form after being dried.

In some aspects, insoluble alpha-glucan can be a graft copolymer such as disclosed in Int. Patent Appl. Publ. No. WO2017/079595 or U.S. Patent Appl. Publ. No. 2019/0185893, which are incorporated herein by reference. Such a graft copolymer comprises dextran (as backbone) and alpha-1,3-glucan (as side chain[s]), where the latter component has been grafted onto the former component; typically, this graft copolymer is produced by using dextran, or alpha-1,2-branched dextran, as a primer for alpha-1,3-glucan synthesis by an alpha-1,3-glucan-producing glucosyltransferase as described above. In some aspects, a graft copolymer comprises about, at least about, or less than about, 10%, 20%, 30%, 35%, 40%, 45%, 50%, 55%, 60%, 65%, 70%, 35-65%, 35-60%, 35-55%, 40-65%, 40-60%, 40-55%, 45-65%, 45-60%, 45-55%, 50-65%, 50-60%, or 50-55% by weight of a dextran backbone, where the balance of the graft copolymer is of alpha-1,3-glucan side chain(s). Alpha-1,3-glucan side chain(s) of an alpha-glucan graft copolymer herein can be alpha-1,3-glucan as presently disclosed (e.g., linkage profile, molecular weight). Dextran backbone of an alpha-glucan graft copolymer herein can comprise about 100% alpha-1,6 glycosidic linkages (i.e., completely linear dextran backbone), or about, or at least about, 90%, 95%, 96%, 97%, 98%, 99%, or 99.5% alpha-1,6 glycosidic linkages (i.e., substantially linear dextran backbone), and/or have a DP or DPw of about, at least about, or less than about, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 85, 90, 95, 100, 105, 110, 150, 200, 250, 300, 400, 500, 8-20, 8-30, 8-100, 8-500, 3-4, 3-5, 3-6, 3-7, 3-8, 4-5, 4-6, 4-7, 4-8, 5-6, 5-7, 5-8, 6-7, 6-8, 7-8, 90-120, 95-120, 100-120, 105-120, 110-120, 115-120, 90-115, 95-115, 100-115, 105-115, 110-115, 90-110, 95-110, 100-110, 105-110, 90-105, 95-105, 100-105, 90-100, 95-100, 90-95, 85-95, or 85-90, for example. In some aspects, a dextran backbone (before being integrated into a graft copolymer) has been alpha-1,2-branched, the percent alpha-1,2 branching of a backbone of a graft copolymer herein can be about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 2-25%, 2-20%, 2-15%, 2-10%, 5-25%, 5-20%, 5-15%, 5-10%, 7-13%, 8-12%, 9-11%, 10-25%, 10-20%, or 10-15%, for example. In some aspects, dextran backbone of an alpha-glucan graft copolymer can comprise (A) (i) about 87-91.5 wt % glucose linked only at positions 1 and 6; (ii) about 0.1-1.2 wt % glucose linked only at positions 1 and 3; (iii) about 0.1-0.7 wt % glucose linked only at positions 1 and 4; (iv) about 7.7-8.6 wt % glucose linked only at positions 1, 3 and 6; and (v) about 0.4-1.7 wt % glucose linked only at: (a) positions 1, 2 and 6, or (b) positions 1, 4 and 6; or (B) (i) about 89.5-90.5 wt % glucose linked only at positions 1 and 6; (ii) about 0.4-0.9 wt % glucose linked only at positions 1 and 3; (iii) about 0.3-0.5 wt % glucose linked only at positions 1 and 4; (iv) about 8.0-8.3 wt % glucose linked only at positions 1, 3 and 6; and (v) about 0.7-1.4 wt % glucose linked only at: (a) positions 1, 2 and 6, or (b) positions 1, 4 and 6. The molecular weight of such a dextran backbone (or any other dextran backbone herein) can be about, or at least about, 0.1, 0.125, 0.15, 0.175, 0.2, 0.24, 0.25, 0.5, 0.75, 1, 2, 3, 4, 5, 6, 7, 8, 9, 10, 20, 30, 40, 50, 60, 70, 80, 90, 100, 110, 120, 130, 140, 150, 160, 170, 180, 190, 200, 0.1-0.2, 0.125-0.175, 0.13-0.17, 0.135-0.165, 0.14-0.16, 0.145-0.155, 10-80, 20-70, 30-60, 40-50, 50-200, 60-200, 70-200, 80-200, 90-200, 100-200, 110-200, 120-200, 50-180, 60-180, 70-180, 80-180, 90-180, 100-180, 110-180, 120-180, 50-160, 60-160, 70-160, 80-160, 90-160, 100-160, 110-160, 120-160, 50-140, 60-140, 70-140, 80-140, 90-140, 100-140, 110-140, 120-140, 50-120, 60-120, 70-120, 80-120, 90-120, 90-110, 100-120, 110-120, 50-110, 60-110, 70-110, 80-110, 90-110, 100-110, 50-100, 60-100, 70-100, 80-100, 90-100, or 95-105 million Daltons, for example. In some aspects in which a graft copolymer is soluble or partly soluble, it can be treated with a dextranase (e.g., any as disclosed in U.S. Patent Appl. Publ. No. 2017/0218093, which is incorporated herein by reference) to remove some of, or all of, the dextran component of the copolymer (e.g., about, or at least about, 20%, 40%, 60%, 70%, 80%, 90%, 95%, or 99% by weight of the dextran is removed) before further processing it to provide a first composition herein.

A dispersion method in some aspects comprises a step of mixing at least aqueous liquid/fluid and the first composition to produce an aqueous dispersion having about 0.5% to about 10% by weight of insoluble alpha-glucan, wherein the mixing comprises (i) applying pressure of at least about 1000 psi, or (ii) applying forces that are substantially equivalent to those forces that are applied in (i). In some alternative aspects, particularly with regard to using a first composition with about 10% to 55% insoluble alpha-glucan by weight of the first composition, a dispersion method comprises a step of mixing at least aqueous liquid/fluid and the first composition to produce an aqueous dispersion having about 0.01% to about 8.5% by weight of insoluble alpha-glucan.

Prior to applying at least about 1000 psi or substantially equivalent forces, at least aqueous liquid (e.g., water or aqueous solution) and a first composition provided in the first step are mixed together. This can entail one or more of stirring, shaking, vortexing, agitation, blending, paddling, rotating, sonication, comminuting, and/or shearing, for example. In some aspects, this first step of mixing can be performed by, or further include, using a sonicator (e.g., ultrasonicator) (e.g., 40-60 W, ˜50 W), homomixer, homogenizer (e.g., rotary or piston, rotar-stator; not high pressure), planetary mixer, colloid mill, jet mill, vortex, and/or any other suitable methodology. Yet, in some aspects, this first step of mixing can be performed by simple means only such as shaking, stirring, or blending, or otherwise by comminuting the insoluble alpha-glucan into the mixture without the aid of a high energy homogenizer or equivalent device. Appropriate amounts of aqueous liquid and first composition herein are mixed such that the mixture (and eventual dispersion resulting from the pressure treatment of at least 1000 psi or substantially equivalent forces) comprises about 0.5% to about 10% (or 0.01% to about 8.5%) by weight of insoluble alpha-glucan (where the balance up to 100 wt % is typically of water or aqueous solution). In some aspects, the mixture and eventual dispersion comprise about, at least about, or no more than about, 0.05, 0.1, 0.5, 1, 2, 3, 4, 5, 6, 7, 7.5, 8, 8.5, 9, 9.5, 10, 0.05-10, 0.05-8, 0.05-6, 0.05-4, 0.05-2, 0.05-1, 1-10, 1-8, 1-6, 1-4, 2-10, 2-8, 2-6, 2-4, 4-10, 4-8, 4-6, 6-10, 6-8, 2-5, 2-4.5, 3-6, 3-5, 3-4.5, 3.5-6, 3.5-5, or 3.5-4.5 wt % of insoluble alpha-glucan (where the balance up to 100 wt % is typically of water or aqueous solution). Aqueous liquid herein for this step can be any as disclosed above, for example. Optionally, one or more other ingredients can be included (e.g., at least one non-soluble component aside from insoluble alpha-glucan), such as an ingredient used to prepare a product (e.g., latex paint) as described below; such addition for mixing can be before or after applying at least about 1000 psi or substantially equivalent forces.

The application of at least about 1000 psi to a mixture comprising at least an aqueous liquid and a first composition of insoluble alpha-glucan herein can be done, for example, by homogenization using a pressure homogenizer (high pressure homogenizer, microfluidizer) to form an aqueous dispersion (colloidal dispersion). Suitable pressure homogenizers include those manufactured by APV GAULIN, DYHYDROMATICS, BEE INTERNATIONAL, GLEN MILLS, RANNIE, and GEA, for example. In some aspects, the pressure applied in homogenization herein can be about, at least about, or no more than about, 1000, 1200, 1400, 1500, 2000, 3000, 4000, 5000, 6000, 7000, 7500, 8000, 8500, 9000, 9500, 10000, 11000, 12000, 13000, 14000, 14500, 15000, 1000-4000, 1000-3000, 1200-4000, 1200-3000, 1400-4000, 1400-3000, 7000-15000, 7000-14500, 7000-12000, 7000-13000, 7000-10000, 7000-9500, 7000-9000, 7000-8500, 7500-15000, 7500-14500, 7500-13000, 7500-12000, 7500-10000, 7500-9500, 7500-9000, 7500-8500, 8000-15000, 8000-14500, 8000-13000, 8000-12000, 8000-10000, 8000-9500, 8000-9000, or 8000-8500 psi. The flow rate of homogenization herein can be about, or at least about, or no more than about, 10, 11, 12, 13, 14, 15, 16, 17, 18, 10-18, 10-17, 10-16, 12-18, 12-17, 12-16, 13-18, 13-17, 13-16, 14-18, 14-17, or 14-16 gph (gallons per hour), for example. Homogenization can be conducted at room temperature or at about, at least about, or no more than about, 15, 20, 25, 30, 35, 40, 45, 50, 55, 60, 65, 70, 15-70, 15-60, 15-50, 15-40, 15-30, 15-25, 20-70, 20-60, 20-50, 20-40, 20-30, or 20-25° C., for example. The number of times a sample is circulated/passed in its entirety (or at least about 95% or 98% of its entirety) through a pressure homogenizer (“passes”) can be 1, 2, 3, 4, 5, 6, 7, 8, 9, 10 or more times, or 1-4, 1-3, 2-4, or 2-3 times, for example. Application of pressure herein at any given time during homogenization typically is against a portion of the dispersion that is in direct contact or proximity to the energy output point/position of the homogenization unit/device; circulation/passing of the dispersion through the unit ensures that the entire (or at least 95% of the) dispersion is treated one or more time with forces generated by the high pressure application. Pressure homogenization as applied in a dispersion method herein can be as described in the Example 1 below, if desired. Forces applied by pressure homogenization herein or a process that applies equivalent forces can optionally comprise one or more of shear (mechanical and/or hydraulic), impaction, turbulence, and/or cavitation.

Typically, an aqueous dispersion method herein does not comprise a step of (i) chemical derivatization (e.g., etherification, esterification, phosphorylation, sulfation, oxidation, xanthation; no substitution of hydrogens of glucan hydroxyl groups with a non-sugar chemical group) of insoluble alpha-glucan; (ii) hydrolysis (e.g., acid-, chemical-, and/or heat-based hydrolysis) of insoluble alpha-glucan; (iii) dissolution of insoluble alpha-glucan, such as dissolution in a strong base (i.e., caustic, pH 11.0; a hydroxide such as NaOH, KOH, or tetraethyl ammonium hydroxide), organic solvent (e.g., organic ionic liquid), N,N-dimethylacetamide (DMAc) (optionally with about 0.5%-5% LiCl), dimethyl sulfoxide (DMSO), N,N-dimethylformamide (DM F), pyridine, SO₂/diethylamine (DEA)/DMSO, LiCl/1,3-dimethyl-2-imidazolidinone (DMI), DMSO/tetrabutyl-ammonium fluoride trihydrate (TBAF), N-methylpyrrolidone, and/or amino oxide (e.g., N-methylmorpholine-N-oxide [NMMO]); (iv) adding a filler (e.g., wood, pulp, or any other solid substance) or plasticizer (e.g., glycerol) to a composition comprising insoluble alpha-glucan (e.g., prior to drying insoluble alpha-glucan); and/or (v) adding a spacer such as a polysaccharide derivative (e.g., cellulose derivative or glucan derivative) or polyol (e.g., an aliphatic polyol such as ethylene glycol, propanetriol, triethylene glycol, polyethylene glycol, or sorbitol; an aromatic polyol such as cyanidin, corilagin, digallic acid, tannic add, or gallic acid) to a composition comprising insoluble alpha-glucan (e.g., prior to drying insoluble alpha-glucan). A spacer herein is a compound that can be deposited between alpha-glucan molecules and thereby prevent strong hydrogen bonds from forming between the alpha-glucan molecules.

An aqueous dispersion herein can be prepared following a dispersion method as presently disclosed. In some aspects, the viscosity of an aqueous dispersion is higher (e.g., about, or at least about, 10%, 25%, 50%, 75%, 100%, 125%, 150%, 165%, 175%, 200%, 225%, 250%, 300%, 50%-300%, 50%-250%, 50%-225%, 50%-200%, 100%-300%, 100%-250%, 100%-225%, 100%-200%, 150%-300%, 150%-250%, 150%-225%, or 150%-200% higher) than the viscosity that the aqueous dispersion had before application of pressure as disclosed. In some aspects, an aqueous dispersion has a viscosity that is about, or at least about, 10%, 25%, 50%, 75%, 100%, 125%, 150%, 165%, 175%, 200%, 225%, 250%, 300%, 50%-300%, 50%-250%, 50%-225%, 50%-200%, 100%-300%, 100%-250%, 100%-225%, 100%-200%, 150%-300%, 150%-250%, 150%-225%, or 150%-200% higher than the viscosity that the aqueous dispersion would have had if it was instead prepared by mixing at an rpm (revolutions per minute) of no more than 11000 (i.e., mixing did not comprise applying pressure of at least 7000 psi, but rather mixing at 11000 rpm or less). Such other mixing can be at an rpm of no more than 3000, 4000, 5000, 6000, 7000, 8000, 9000, 10000, or 11000, for example. Such other mixing can be applied for a time of about, or no more than about, 2, 3, 4, 5, 6, 7, 8, 9, 10, 11, 12, 13, 14, 15, or 16 minutes, for example. Such other mixing can be with a disperser (e.g., ULTRATURRAX, IKA, Wilmington, N.C.), sonicator, homogenizer (non-pressure) (e.g., rotor stator such as IKA DR 2000/20), colloid mill (e.g., IKA MK 2000/20), jet mill, wet grinding mill (e.g. Dyno Mill), or bead mill for example. In some alternative aspects, particularly with aqueous dispersions produced by dispersing insoluble alpha-glucan that has never been dried (using a dispersal technique as disclosed herein), an aqueous dispersion has a viscosity that is about, or at least about, 200%, 300%, 400%, 500%, 600%, 700%, 800%, 900%, 1000%, 1100%, 1200%, 200%-1200%, 200%-1000%, 400%-1200%, or 400%-1000% higher than the viscosity that the aqueous dispersion would have had if it was instead prepared by mixing at an rpm of no more than 11000 (e.g., or an rpm listed above).

The viscosity of an aqueous dispersion herein (or a composition comprising such an aqueous dispersion) can be about, or at least about, 30, 35, 40, 45, 50, or 60 centipoise (cps), for example. In some alternative aspects, particularly with an aqueous dispersion produced by dispersing insoluble alpha-glucan that has never been dried (using a dispersal technique as disclosed herein), the viscosity of the aqueous dispersion can be about, or at least about, 100, 125, 150, or 175 cps. Viscosity herein can be as measured with an aqueous dispersion at any temperature between about 3° C. to about 80° C., for example (e.g., 4-30° C., 15-30° C., 15-25° C.). Viscosity typically is as measured at atmospheric pressure (about 760 torr) or a pressure that is ±10% thereof. Viscosity can be measured using a viscometer or rheometer, for example, and can optionally be as measured at a shear rate (rotational shear rate) of about 0.1, 0.5, 1.0, 1.667, 2, 5, 10, 50, 100, 500, 1000, 0.1-500, 0.1-100, 1.0-500, 1.0-1000, or 1.0-100 s⁻¹ (1/s), for example. Viscosity can optionally be measured following the procedure outlined in the below Examples.

In some aspects of the present disclosure, at least about 60% by weight of the insoluble alpha-glucan particles of an aqueous dispersion have a diameter of less than about 30 micrometers (microns). Yet, in some aspects, about, or at least about, 60%, 65%, 70%, 75%, 60-75%, 60-70%, 60-65%, 65-75%, or 65-70% by weight of the insoluble alpha-glucan particles of an aqueous dispersion have a diameter of about, or less than about, 25, 30, 35, 40, 25-40, 30-40, 35-40, 25-35, or 30-35 micrometers. In some aspects, about 40-60%, 40-55%, 45-60%, 45-55%, 47-53%, 48-52%, 49-51%, or 50% by weight of the insoluble alpha-glucan particles of an aqueous dispersion have a diameter of about, or less than about, 15, 16, 16, 18, 19, 20, 21, 22, 15-22, 15-20, 15-18, 16-22, 16-20, or 16-18 micrometers. In some alternative aspects, particularly with insoluble alpha-glucan particles produced by dispersing insoluble alpha-glucan that has never been dried (using a dispersal technique as disclosed herein), at least about 90%, 95%, 96%, 97%, 98%, 99%, or 100% by weight of the insoluble alpha-glucan particles of an aqueous dispersion have a diameter of less than about 30, 35, 40, 45, or 50 micrometers. Particle size herein can be measured by a process comprising light scattering or electrical impedance change (e.g., using a Coulter Counter), as described in any of U.S. Pat. Nos. 6,091,492, 6,741,350 and 9,297,737 (all incorporated herein by reference), and/or as disclosed in the below Examples, for example.

It is notable that aqueous dispersions of insoluble alpha-glucan herein typically have enhanced stability in that the particles of alpha-glucan are able to remain dispersed following formation of the dispersion. For example, in an aqueous dispersion comprising insoluble alpha-glucan herein, the insoluble alpha-glucan particles are dispersed through about, or at least about, 55%, 60%, 65%, 70%, 75%, 80%, 55%-80%, 55%-75%, 55%-70%, 55%-65%, 60%-80%, 60%-75%, 60%-70%, 60%-65%, 65%-80%, 65%-75%, or 65%-70% of the volume of the dispersion. In some alternative aspects, particularly with insoluble alpha-glucan particles produced by dispersing insoluble alpha-glucan that has never been dried (using a dispersal technique as disclosed herein), the insoluble alpha-glucan particles are dispersed through about, or at least about, 80%, 85%, 90%, 95%, 96%, 97%, 98%, 99%, 80%-99%, 80%-96%, 85%-99%, 85%-96%, 90%-99%, or 90%-96% of the volume of the dispersion. In some aspects, any of the above levels of dispersion is contemplated to be for a time (typically beginning from initial preparation of the dispersion) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years, optionally at a temperature of about, or up to about, 15, 20, 25, 30, 35, 40, 50, 60, 70, or 80° C., and/or at a pH of about 4, 5, 6, 7, 8, 9, or 4-9. In some aspects, stability can additionally or alternatively refer to the insoluble alpha-glucan herein having an enhanced ability to provide viscosity to an aqueous composition (e.g., any of the above viscosity levels, optionally for any of the above time periods). In some aspects, dispersion of insoluble alpha-glucan particles in an emulsion confers stability to the emulsion; for example, any of the above dispersal-volume percentages and/or times of such stability can likewise characterize dispersed/emulsified droplets.

In some aspects, the homogeneity (e.g., as reflected by a dispersion's viscosity and/or capacity to remain dispersed [stability], as above) of insoluble alpha-glucan particles in an aqueous dispersion herein is higher (e.g., about, or at least about, 10%, 25%, 50%, 75%, 100%, 125%, 150%, 165%, 175%, 200%, 225%, 250%, 300%, 50%-300%, 50%-250%, 50%-225%, 50%-200%, 100%-300%, 100%-250%, 100%-225%, 100%-200%, 150%-300%, 150%-250%, 150%-225%, or 150%-200% higher) than the homogeneity that the aqueous dispersion had before application of pressure as disclosed herein.

An aqueous dispersion herein can comprise about, at least about, or no more than about, 0.5, 1, 2, 3, 4, 5, 6, 7, 8, 9, 10, 0.05-10, 0.05-8, 0.05-6, 0.05-4, 0.05-2, 0.05-1, 1-10, 1-8, 1-6, 1-4, 2-10, 2-8, 2-6, 2-4, 4-10, 4-8, 4-6, 6-10, 6-8, 2-5, 2-4.5, 3-6, 3-5, 3-4.5, 3.5-6, 3.5-5, or 3.5-4.5 wt % insoluble alpha-glucan (where the balance up to 100 wt % is typically of water or aqueous solution), for example. In some alternative aspects, the insoluble alpha-glucan used for preparing such an aqueous dispersion can be that which was never-dried (e.g., comprising about 10-50 wt % insoluble alpha-glucan before dispersal, and the balance is of water/aqueous solution [an example is insoluble alpha-glucan produced in situ in a product such as a food), instead of insoluble alpha-glucan that has been dried.

An aqueous dispersion herein can be a product of a dispersion method as presently disclosed, and thus have any of the features of such a product. In some aspects, an aqueous dispersion can comprise about 0.5 wt % to about 10 wt % insoluble alpha-glucan herein, wherein: at least 60% by weight of the insoluble alpha-glucan particles in the aqueous dispersion have a diameter of less than 30 micrometers, the insoluble alpha-glucan is dispersed through at least about 60% of the volume of the aqueous dispersion, and at least 50% of the glycosidic linkages of the insoluble alpha-glucan are alpha-1,3 glycosidic linkages. Yet, in some alternative aspects, particularly with aqueous dispersions produced by dispersing insoluble alpha-glucan that has never been dried (using a dispersal technique as disclosed herein), an aqueous dispersion can comprise about 0.01 wt % to about 8.5 wt % insoluble alpha-glucan herein, wherein: at least 90% by weight of the insoluble alpha-glucan particles in the aqueous dispersion have a diameter of less than 30 micrometers, the insoluble alpha-glucan is dispersed through at least about 80% of the volume of the aqueous dispersion, and at least 50% of the glycosidic linkages of the insoluble alpha-glucan are alpha-1,3 glycosidic linkages.

A composition comprising an aqueous dispersion of insoluble alpha-glucan herein can, in some aspects, comprise one or more salts such as a sodium salt (e.g., NaCl, Na₂SO₄). Other non-limiting examples of salts include those having (i) an aluminum, ammonium, barium, calcium, chromium (II or III), copper (I or II), iron (II or III), hydrogen, lead (II), lithium, magnesium, manganese (II or III), mercury (I or II), potassium, silver, sodium strontium, tin (II or IV), or zinc cation, and (ii) an acetate, borate, bromate, bromide, carbonate, chlorate, chloride, chlorite, chromate, cyanamide, cyanide, dichromate, dihydrogen phosphate, ferricyanide, ferrocyanide, fluoride, hydrogen carbonate, hydrogen phosphate, hydrogen sulfate, hydrogen sulfide, hydrogen sulfite, hydride, hydroxide, hypochlorite, iodate, iodide, nitrate, nitride, nitrite, oxalate, oxide, perchlorate, permanganate, peroxide, phosphate, phosphide, phosphite, silicate, stannate, stannite, sulfate, sulfide, sulfite, tartrate, or thiocyanate anion. Thus, any salt having a cation from (i) above and an anion from (ii) above can be in a composition, for example. A salt can be present in an aqueous dispersion herein at a wt % of about, or at least about, 0.01, 0.025, 0.05, 0.075, 0.1, 0.25, 0.5, 0.75, 1.0, 1.25, 1.5, 1.75, 2.0, 2.5, 3.0, 3.5, 0.01-3.5, 0.5-3.5, 0.5-2.5, or 0.5-1.5 wt % (such wt % values typically refer to the total concentration of one or more salts), for example.

A composition comprising an aqueous dispersion of insoluble alpha-glucan herein can optionally contain one or more active enzymes. Examples of suitable enzymes include proteases, cellulases, hemicellulases, peroxidases, lipolytic enzymes (e.g., metallolipolytic enzymes), xylanases, lipases, phospholipases, esterases (e.g., arylesterase, polyesterase), perhydrolases, cutinases, pectinases, pectate lyases, mannanases, keratinases, reductases, oxidases (e.g., choline oxidase), phenoloxidases, lipoxygenases, ligninases, pullulanases, tannases, pentosanases, malanases, beta-glucanases, arabinosidases, hyaluronidases, chondroitinases, laccases, metalloproteinases, amadoriases, glucoamylases, arabinofuranosidases, phytases, isomerases, transferases, nucleases and amylases. If an enzyme(s) is included, it may be comprised in a composition herein at about 0.0001-0.1 wt % (e.g., 0.01-0.03 wt %) active enzyme (e.g., calculated as pure enzyme protein), for example. In fabric care applications, an enzyme (e.g., any of the above such as cellulase) can be present in an aqueous composition in which a fabric is treated (e.g., wash liquor) at a concentration that is minimally about 0.01-0.1 ppm total enzyme protein, or about 0.1-10 ppb total enzyme protein (e.g., less than 1 ppm), to maximally about 100, 200, 500, 1000, 2000, 3000, 4000, or 5000 ppm total enzyme protein, for example.

A composition comprising an aqueous dispersion of insoluble alpha-glucan herein can be in the form of a household care product, personal care product, industrial product, ingestible product (e.g., food product), or pharmaceutical product, for example. Examples of such products can be as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are all incorporated herein by reference. In some aspects, a composition can further comprise at least one component/ingredient of a household care product, personal care product, industrial product, pharmaceutical product, or ingestible product (e.g., food product) as disclosed in any of the foregoing publications and/or as presently disclosed.

Insoluble alpha-glucan disclosed herein are believed to be useful for providing one or more of the following physical properties to a personal care product, pharmaceutical product, household product, industrial product, or ingestible product (e.g., food product): thickening, freeze/thaw stability, lubricity, moisture retention and release, texture, consistency, shape retention, emulsification, binding, suspension, dispersion, gelation, reduced mineral hardness, for example. Examples of a concentration or amount of insoluble alpha-glucan in a product can be any of the weight percentages provided herein, for example.

Personal care products herein are not particularly limited and include, for example, skin care compositions, cosmetic compositions, antifungal compositions, and antibacterial compositions. Personal care products herein may be in the form of, for example, lotions, creams, pastes, balms, ointments, pomades, gels, liquids, combinations of these and the like. The personal care products disclosed herein can include at least one active ingredient, if desired. An active ingredient is generally recognized as an ingredient that causes an intended cosmetic or pharmacological effect.

In certain embodiments, a skin care product can be applied to skin for addressing skin damage related to a lack of moisture. A skin care product may also be used to address the visual appearance of skin (e.g., reduce the appearance of flaky, cracked, and/or red skin) and/or the tactile feel of the skin (e.g., reduce roughness and/or dryness of the skin while improved the softness and subtleness of the skin). A skin care product typically may include at least one active ingredient for the treatment or prevention of skin ailments, providing a cosmetic effect, or for providing a moisturizing benefit to skin, such as zinc oxide, petrolatum, white petrolatum, mineral oil, cod liver oil, lanolin, dimethicone, hard fat, vitamin A, allantoin, calamine, kaolin, glycerin, or colloidal oatmeal, and combinations of these. A skin care product may include one or more natural moisturizing factors such as ceramides, hyaluronic acid, glycerin, squalane, amino acids, cholesterol, fatty acids, triglycerides, phospholipids, glycosphingolipids, urea, linoleic acid, glycosaminoglycans, mucopolysaccharide, sodium lactate, or sodium pyrrolidone carboxylate, for example. Other ingredients that may be included in a skin care product include, without limitation, glycerides, apricot kernel oil, canola oil, squalane, squalene, coconut oil, corn oil, jojoba oil, jojoba wax, lecithin, olive oil, safflower oil, sesame oil, shea butter, soybean oil, sweet almond oil, sunflower oil, tea tree oil, shea butter, palm oil, cholesterol, cholesterol esters, wax esters, fatty acids, aloe vera and orange oil.

A personal care product herein can also be in the form of makeup, lipstick, mascara, rouge, foundation, blush, eyeliner, lip liner, lip gloss, other cosmetics, sunscreen, sun block, nail polish, nail conditioner, bath gel, shower gel, body wash, face wash, lip balm, skin conditioner, cold cream, moisturizer, body spray, soap, body scrub, exfoliant, astringent, scruffing lotion, depilatory, permanent waving solution, antidandruff formulation, antiperspirant composition, deodorant, shaving product, pre-shaving product, after-shaving product, cleanser, skin gel, rinse, dentifrice composition, toothpaste, or mouthwash, for example. An example of a personal care product (e.g., a cleanser, soap, scrub, cosmetic) comprises a carrier or exfoliation agent (e.g., jojoba beads [jojoba ester beads]) (e.g., about 1-10, 3-7, 4-6, or 5 wt %); such an agent may optionally be dispersed within the product.

A personal care product in some aspects can be a hair care product. Examples of hair care products herein include shampoo, hair conditioner (leave-in or rinse-out), cream rinse, hair dye, hair coloring product, hair shine product, hair serum, hair anti-frizz product, hair split-end repair product, mousse, hair spray, and styling gel. A hair care product can be in the form of a liquid, paste, or gel in some embodiments. A hair care product as presently disclosed typically comprises one or more of the following ingredients, which are generally used to formulate hair care products: anionic surfactants such as polyoxyethylenelauryl ether sodium sulfate; cationic surfactants such as stearyltrimethylammonium chloride and/or distearyltrimethylammonium chloride; nonionic surfactants such as glyceryl monostearate, sorbitan monopalmitate and/or polyoxyethylenecetyl ether; wetting agents such as propylene glycol, 1,3-butylene glycol, glycerin, sorbitol, pyroglutamic acid salts, amino acids and/or trimethylglycine, hydrocarbons such as liquid paraffins, petrolatum, solid paraffins, squalane and/or olefin oligomers; higher alcohols such as stearyl alcohol and/or cetyl alcohol; superfatting agents; antidandruff agents; disinfectants; anti-inflammatory agents; crude drugs; water-soluble polymers such as methyl cellulose, hydroxycellulose and/or partially deacetylated chitin; antiseptics such as paraben; ultra-violet light absorbers; pearling agents; pH adjustors; perfumes; and pigments.

A pharmaceutical product herein can be in the form of an emulsion, liquid (e.g., as comprised in an ampoule or liquid capsule), elixir, gel, suspension, solution, cream, or ointment, for example. Also, a pharmaceutical product herein can be in the form of any of the personal care products disclosed herein, such as an antibacterial or antifungal composition. A pharmaceutical product can further comprise one or more pharmaceutically acceptable carriers, diluents, and/or pharmaceutically acceptable salts.

A household and/or industrial product herein can be in the form of drywall tape-joint compounds; mortars; grouts; cement plasters; spray plasters; cement stucco; adhesives; pastes; wall/ceiling texturizers; binders and processing aids for tape casting, extrusion forming, injection molding and ceramics; spray adherents and suspending/dispersing aids for pesticides, herbicides, and fertilizers; fabric care products such as fabric softeners and laundry detergents; hard surface cleaners; air fresheners; polymer emulsions; latex; gels such as water-based gels; surfactant solutions; paints such as water-based paints; protective coatings; adhesives; sealants and caulks; inks such as water-based ink; metal-working fluids; fluids for preparing films or coatings; or emulsion-based metal cleaning fluids used in electroplating, phosphatizing, galvanizing and/or general metal cleaning operations, for example.

Compositions disclosed herein can be in the form of a detergent composition such as a fabric care composition. A fabric care composition herein can be used for hand wash, machine wash and/or other purposes such as soaking and/or pretreatment of fabrics, for example. A fabric care composition may take the form of, for example, a laundry detergent; fabric conditioner; any wash-, rinse-, or dryer-added product; unit dose or spray. Fabric care compositions in a liquid form may be in the form of an aqueous composition as disclosed herein. Other non-limiting examples of fabric care compositions herein include: liquid, gel or paste-form all-purpose or heavy-duty washing agents; liquid or dry fine-fabric (e.g. delicates) detergents; cleaning auxiliaries such as bleach additives, “stain-stick”, or pre-treatments; substrate-laden products such as wetted wipes, pads, or sponges; sprays and mists.

A detergent composition herein may be in any useful form such as a paste, unit dose, or liquid. A liquid detergent may be aqueous, typically containing up to about 70 wt % of water and 0 wt % to about 30 wt % of organic solvent. It may also be in the form of a compact gel type containing only about 30 wt % water.

A detergent composition herein typically comprises one or more surfactants, wherein the surfactant is selected from nonionic surfactants, anionic surfactants, cationic surfactants, ampholytic surfactants, zwitterionic surfactants, semi-polar nonionic surfactants and mixtures thereof. In some embodiments, the surfactant is present at a level of from about 0.1% to about 60%, while in alternative embodiments the level is from about 1% to about 50%, while in still further embodiments the level is from about 5% to about 40%, by weight of the detergent composition. A detergent will usually contain 0 wt % to about 50 wt % of an anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. In addition, a detergent composition may optionally contain 0 wt % to about 40 wt % of a nonionic surfactant such as alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, or polyhydroxy alkyl fatty acid amide (as described for example in WO92/06154, which is incorporated herein by reference).

A detergent composition herein typically comprises one or more detergent builders or builder systems. In some aspects, oxidized alpha-1,3-glucan can be included as a co-builder, in which it is used together with one or more additional builders such as any disclosed herein. Oxidized alpha-1,3-glucan compounds for use herein are disclosed in U.S. Patent Appl. Publ. No. 2015/0259439. In some embodiments incorporating at least one builder, the cleaning compositions comprise at least about 1%, from about 3% to about 60%, or even from about 5% to about 40%, builder by weight of the composition. Builders (in addition to oxidized alpha-1,3-glucan) include, but are not limited to, alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1, 3, 5-trihydroxy benzene-2, 4, 6-trisulphonic acid, and carboxymethyloxysuccinic acid, various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure. Additional examples of a detergent builder or complexing agent include zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g., SKS-6 from Hoechst).

In some embodiments, builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (See, e.g., EP2100949).

In some embodiments, suitable builders can include phosphate builders and non-phosphate builders. In some embodiments, a builder is a phosphate builder. In some embodiments, a builder is a non-phosphate builder. A builder can be used in a level of from 0.1% to 80%, or from 5% to 60%, or from 10% to 50%, by weight of the composition. In some embodiments, the product comprises a mixture of phosphate and non-phosphate builders. Suitable phosphate builders include mono-phosphates, di-phosphates, tri-polyphosphates or oligomeric-polyphosphates, including the alkali metal salts of these compounds, including the sodium salts. In some embodiments, a builder can be sodium tripolyphosphate (STPP). Additionally, the composition can comprise carbonate and/or citrate, preferably citrate that helps to achieve a neutral pH composition. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include ammonium and/or alkali metal salts, i.e., lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

A detergent composition herein can comprise at least one chelating agent. Suitable chelating agents include, but are not limited to copper, iron and/or manganese chelating agents and mixtures thereof. In embodiments in which at least one chelating agent is used, the composition comprises from about 0.1% to about 15%, or even from about 3.0% to about 10%, chelating agent by weight of the composition.

A detergent composition herein can comprise at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

A detergent composition herein can comprise one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Additional dye transfer inhibiting agents include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP), hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetraacetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethyl ethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof, which can be used alone or in combination with any of the above. In embodiments in which at least one dye transfer inhibiting agent is used, a composition herein may comprise from about 0.0001% to about 10%, from about 0.01% to about 5%, or even from about 0.1% to about 3%, by weight of the composition.

A detergent composition herein can comprise silicates. In some of these embodiments, sodium silicates (e.g., sodium disilicate, sodium metasilicate, and/or crystalline phyllosilicates) find use. In some embodiments, silicates are present at a level of from about 1% to about 20% by weight of the composition. In some embodiments, silicates are present at a level of from about 5% to about 15% by weight of the composition.

A detergent composition herein can comprise dispersants. Suitable water-soluble organic materials include, but are not limited to the homo- or co-polymeric acids or their salts, in which the polycarboxylic acid comprises at least two carboxyl radicals separated from each other by not more than two carbon atoms.

A detergent composition herein may additionally comprise one or more enzymes as disclosed above, for example. In some aspects, a detergent composition can comprise one or more enzymes, each at a level from about 0.00001% to about 10% by weight of the composition and the balance of cleaning adjunct materials by weight of composition. In some other aspects, a detergent composition can also comprise each enzyme at a level of about 0.0001% to about 10%, about 0.001% to about 5%, about 0.001% to about 2%, or about 0.005% to about 0.5%, by weight of the composition. Enzymes comprised in a detergent composition herein may be stabilized using conventional stabilizing agents, e.g., a polyol such as propylene glycol or glycerol; a sugar or sugar alcohol; lactic acid; boric acid or a boric acid derivative (e.g., an aromatic borate ester).

A detergent composition in some aspects can comprise one or more other types of polymer in addition to insoluble alpha-glucan as disclosed herein. Examples of other polymers useful herein include carboxymethyl cellulose (CMC), dextran, poly(vinylpyrrolidone) (PVP), polyethylene glycol (PEG), poly(vinyl alcohol) (PVA), polycarboxylates such as polyacrylates, maleic/acrylic acid copolymers and lauryl methacrylate/acrylic acid copolymers.

A detergent composition herein may contain a bleaching system. For example, a bleaching system can comprise an H₂O₂ source such as perborate or percarbonate, which may be combined with a peracid-forming bleach activator such as tetraacetylethylenediamine (TAED) or nonanoyloxybenzenesulfonate (NOBS). Alternatively, a bleaching system may comprise peroxyacids (e.g., amide, imide, or sulfone type peroxyacids). Alternatively still, a bleaching system can be an enzymatic bleaching system comprising perhydrolase, for example, such as the system described in WO2005/056783.

A detergent composition herein may also contain conventional detergent ingredients such as fabric conditioners, clays, foam boosters, suds suppressors, anti-corrosion agents, soil-suspending agents, anti-soil redeposition agents, dyes, bactericides, tarnish inhibiters, optical brighteners, or perfumes. The pH of a detergent composition herein (measured in aqueous solution at use concentration) is usually neutral or alkaline (e.g., pH of about 7.0 to about 11.0).

It is believed that insoluble alpha-glucan herein can be included as an anti-redeposition agent and/or clay soil removal agent in a detergent composition such as a fabric care composition, if desired (such agents can optionally be characterized as whiteness maintenance agents in certain aspects). Examples of other suitable anti-redeposition and/or clay soil removal agents herein include polyethoxy zwitterionic surfactants, water-soluble copolymers of acrylic or methacrylic acid with acrylic or methacrylic acid-ethylene oxide condensates (e.g., U.S. Pat. No. 3,719,647), cellulose derivatives such as carboxymethylcellulose and hydroxypropylcellulose (e.g., U.S. Pat. Nos. 3,597,416 and 3,523,088), and mixtures comprising nonionic alkyl polyethoxy surfactant, polyethoxy alkyl quaternary cationic surfactant and fatty amide surfactant (e.g., U.S. Pat. No. 4,228,044). Non-limiting examples of other suitable anti-redeposition and clay soil removal agents are disclosed in U.S. Pat. Nos. 4,597,898 and 4,891,160, and International Patent Appl. Publ. No. WO95/32272, all of which are incorporated herein by reference.

Particular forms of detergent compositions that can be adapted for purposes disclosed herein are disclosed in, for example, US20090209445A1, US20100081598A1, U.S. Pat. No. 7,001,878B2, EP1504994B1, WO2001085888A2, WO2003089562A1, WO2009098659A1, WO2009098660A1, WO2009112992A1, WO2009124160A1, WO2009152031A1, WO2010059483A1, WO2010088112A1, WO2010090915A1, WO2010135238A1, WO2011094687A1, WO2011094690A1, WO2011127102A1, WO2011163428A1, WO2008000567A1, WO2006045391A1, WO2006007911A1, WO2012027404A1, EP1740690B1, WO2012059336A1, U.S. Pat. No. 6,730,646B1, WO2008087426A1, WO2010116139A1, and WO2012104613A1, all of which are incorporated herein by reference.

Laundry detergent compositions herein can optionally be heavy duty (all purpose) laundry detergent compositions. Exemplary heavy duty laundry detergent compositions comprise a detersive surfactant (10%-40% wt/wt), including an anionic detersive surfactant (selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof), and optionally non-ionic surfactant (selected from a group of linear or branched or random chain, substituted or unsubstituted alkyl alkoxylated alcohol, e.g., C8-C18 alkyl ethoxylated alcohols and/or C6-C12 alkyl phenol alkoxylates), where the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from a group of alkyl pyridinium compounds, alkyl quaternary ammonium compounds, alkyl quaternary phosphonium compounds, alkyl ternary sulphonium compounds, and/or mixtures thereof); zwitterionic and/or amphoteric detersive surfactants (selected from a group of alkanolamine sulpho-betaines); ampholytic surfactants; semi-polar non-ionic surfactants and mixtures thereof.

A detergent herein such as a heavy duty laundry detergent composition may optionally include, a surfactancy boosting polymer consisting of amphiphilic alkoxylated grease cleaning polymers (selected from a group of alkoxylated polymers having branched hydrophilic and hydrophobic properties, such as alkoxylated polyalkylenimines in the range of 0.05 wt %-10 wt %) and/or random graft polymers (typically comprising of hydrophilic backbone comprising monomers selected from the group consisting of: unsaturated C1-C6 carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C4-C25 alkyl group, polypropylene, polybutylene, vinyl ester of a saturated C1-C6 mono-carboxylic acid, C1-C6 alkyl ester of acrylic or methacrylic acid, and mixtures thereof.

A detergent herein such as a heavy duty laundry detergent composition may optionally include additional polymers such as soil release polymers (include anionically end-capped polyesters, for example SRP1, polymers comprising at least one monomer unit selected from saccharide, dicarboxylic acid, polyol and combinations thereof, in random or block configuration, ethylene terephthalate-based polymers and co-polymers thereof in random or block configuration, for example REPEL-O-TEX SF, SF-2 AND SRP6, TEXCARE SRA100, SRA300, SRN100, SRN170, SRN240, SRN300 AND SRN325, MARLOQUEST SL), anti-redeposition agent(s) herein (0.1 wt % to 10 wt %), include carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof, vinylpyrrolidone homopolymer, and/or polyethylene glycol, molecular weight in the range of from 500 to 100,000 Da); and polymeric carboxylate (such as maleate/acrylate random copolymer or polyacrylate homopolymer).

A detergent herein such as a heavy duty laundry detergent composition may optionally further include saturated or unsaturated fatty acids, preferably saturated or unsaturated C12-C24 fatty acids (0 wt % to 10 wt %); deposition aids (examples for which include polysaccharides, cellulosic polymers, poly diallyl dimethyl ammonium halides (DADMAC), and co-polymers of DAD MAC with vinyl pyrrolidone, acrylamides, imidazoles, imidazolinium halides, and mixtures thereof, in random or block configuration, cationic guar gum, cationic starch, cationic polyacrylamides, and mixtures thereof.

A detergent herein such as a heavy duty laundry detergent composition may optionally further include dye transfer inhibiting agents, examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents, examples of which include ethylene-diamine-tetraacetic acid (EDTA), diethylene triamine penta methylene phosphonic acid (DTPMP), hydroxy-ethane diphosphonic acid (HEDP), ethylenediamine N,N′-disuccinic acid (EDDS), methyl glycine diacetic acid (MGDA), diethylene triamine penta acetic acid (DTPA), propylene diamine tetraacetic acid (PDTA), 2-hydroxypyridine-N-oxide (HPNO), or methyl glycine diacetic acid (MGDA), glutamic acid N,N-diacetic acid (N,N-dicarboxymethyl glutamic acid tetrasodium salt (GLDA), nitrilotriacetic acid (NTA), 4,5-dihydroxy-m-benzenedisulfonic acid, citric acid and any salts thereof, N-hydroxyethylethylenediaminetriacetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

A detergent herein such as a heavy duty laundry detergent composition may optionally include silicone or fatty-acid based suds suppressors; hueing dyes, calcium and magnesium cations, visual signaling ingredients, anti-foam (0.001 wt % to about 4.0 wt %), and/or a structurant/thickener (0.01 wt % to 5 wt %) selected from the group consisting of diglycerides and triglycerides, ethylene glycol distearate, microcrystalline cellulose, microfiber cellulose, biopolymers, xanthan gum, gellan gum, and mixtures thereof). Such structurant/thickener would be, in some aspects, in addition to the insoluble alpha-glucan comprised in the detergent. A structurant can also be referred to as a structural agent.

A detergent herein can be in the form of a heavy duty dry/solid laundry detergent composition, for example. Such a detergent may include: (i) a detersive surfactant, such as any anionic detersive surfactant disclosed herein, any non-ionic detersive surfactant disclosed herein, any cationic detersive surfactant disclosed herein, any zwitterionic and/or amphoteric detersive surfactant disclosed herein, any ampholytic surfactant, any semi-polar non-ionic surfactant, and mixtures thereof; (ii) a builder, such as any phosphate-free builder (e.g., zeolite builders in the range of 0 wt % to less than 10 wt %), any phosphate builder (e.g., sodium tri-polyphosphate in the range of 0 wt % to less than 10 wt %), citric acid, citrate salts and nitrilotriacetic acid, any silicate salt (e.g., sodium or potassium silicate or sodium meta-silicate in the range of 0 wt % to less than 10 wt %); any carbonate salt (e.g., sodium carbonate and/or sodium bicarbonate in the range of 0 wt % to less than 80 wt %), and mixtures thereof; (iii) a bleaching agent, such as any photobleach (e.g., sulfonated zinc phthalocyanines, sulfonated aluminum phthalocyanines, xanthenes dyes, and mixtures thereof), any hydrophobic or hydrophilic bleach activator (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, nonanoyloxybenzene sulfonate-NOBS, nitrile quats, and mixtures thereof), any source of hydrogen peroxide (e.g., inorganic perhydrate salts, examples of which include mono or tetra hydrate sodium salt of perborate, percarbonate, persulfate, perphosphate, or persilicate), any preformed hydrophilic and/or hydrophobic peracids (e.g., percarboxylic acids and salts, percarbonic acids and salts, perimidic acids and salts, peroxymonosulfuric acids and salts, and mixtures thereof); and/or (iv) any other components such as a bleach catalyst (e.g., imine bleach boosters examples of which include iminium cations and polyions, iminium zwitterions, modified amines, modified amine oxides, N-sulphonyl imines, N-phosphonyl imines, N-acyl imines, thiadiazole dioxides, perfluoroimines, cyclic sugar ketones, and mixtures thereof), and a metal-containing bleach catalyst (e.g., copper, iron, titanium, ruthenium, tungsten, molybdenum, or manganese cations along with an auxiliary metal cations such as zinc or aluminum and a sequestrate such as EDTA, ethylenediaminetetra(methylenephosphonic acid).

Compositions disclosed herein can be in the form of a dishwashing detergent composition, for example. Examples of dishwashing detergents include automatic dishwashing detergents (typically used in dishwasher machines) and hand-washing dish detergents. A dishwashing detergent composition can be in any dry or liquid/aqueous form as disclosed herein, for example. Components that may be included in certain embodiments of a dishwashing detergent composition include, for example, one or more of a phosphate; oxygen- or chlorine-based bleaching agent; non-ionic surfactant; alkaline salt (e.g., metasilicates, alkali metal hydroxides, sodium carbonate); any active enzyme disclosed herein; anti-corrosion agent (e.g., sodium silicate); anti-foaming agent; additives to slow down the removal of glaze and patterns from ceramics; perfume; anti-caking agent (in granular detergent); starch (in tablet-based detergents); gelling agent (in liquid/gel based detergents); and/or sand (powdered detergents).

Dishwashing detergents such as an automatic dishwasher detergent or liquid dishwashing detergent can comprise (i) a non-ionic surfactant, including any ethoxylated non-ionic surfactant, alcohol alkoxylated surfactant, epoxy-capped poly(oxyalkylated) alcohol, or amine oxide surfactant present in an amount from 0 to 10 wt %; (ii) a builder, in the range of about 5-60 wt %, including any phosphate builder (e.g., mono-phosphates, di-phosphates, tri-polyphosphates, other oligomeric-polyphosphates, sodium tripolyphosphate-STPP), any phosphate-free builder (e.g., amino acid-based compounds including methyl-glycine-diacetic acid [MGDA] and salts or derivatives thereof, glutamic-N,N-diacetic acid [GLDA] and salts or derivatives thereof, iminodisuccinic acid (IDS) and salts or derivatives thereof, carboxy methyl inulin and salts or derivatives thereof, nitrilotriacetic acid [NTA], diethylene triamine penta acetic acid [DTPA], B-alaninediacetic acid [B-ADA] and salts thereof), homopolymers and copolymers of poly-carboxylic acids and partially or completely neutralized salts thereof, monomeric polycarboxylic acids and hydroxycarboxylic acids and salts thereof in the range of 0.5 wt % to 50 wt %, or sulfonated/carboxylated polymers in the range of about 0.1 wt % to about 50 wt %; (iii) a drying aid in the range of about 0.1 wt % to about 10 wt % (e.g., polyesters, especially anionic polyesters, optionally together with further monomers with 3 to 6 functionalities—typically acid, alcohol or ester functionalities which are conducive to polycondensation, polycarbonate-, polyurethane- and/or polyurea-polyorganosiloxane compounds or precursor compounds thereof, particularly of the reactive cyclic carbonate and urea type); (iv) a silicate in the range from about 1 wt % to about 20 wt % (e.g., sodium or potassium silicates such as sodium disilicate, sodium meta-silicate and crystalline phyllosilicates); (v) an inorganic bleach (e.g., perhydrate salts such as perborate, percarbonate, perphosphate, persulfate and persilicate salts) and/or an organic bleach (e.g., organic peroxyacids such as diacyl- and tetraacylperoxides, especially diperoxydodecanedioic acid, diperoxytetradecanedioic acid, and diperoxyhexadecanedioic acid); (vi) a bleach activator (e.g., organic peracid precursors in the range from about 0.1 wt % to about 10 wt %) and/or bleach catalyst (e.g., manganese triazacyclononane and related complexes; Co, Cu, Mn, and Fe bispyridylamine and related complexes; and pentamine acetate cobalt(III) and related complexes); (vii) a metal care agent in the range from about 0.1 wt % to 5 wt % (e.g., benzatriazoles, metal salts and complexes, and/or silicates); and/or (viii) any active enzyme disclosed herein in the range from about 0.01 to 5.0 mg of active enzyme per gram of automatic dishwashing detergent composition, and an enzyme stabilizer component (e.g., oligosaccharides, polysaccharides, and inorganic divalent metal salts).

Compositions disclosed herein can be in the form of an oral care composition, for example. Examples of oral care compositions include dentifrices, toothpaste, mouth wash, mouth rinse, chewing gum, and edible strips that provide some form of oral care (e.g., treatment or prevention of cavities [dental caries], gingivitis, plaque, tartar, and/or periodontal disease). An oral care composition can also be for treating an “oral surface”, which encompasses any soft or hard surface within the oral cavity including surfaces of the tongue, hard and soft palate, buccal mucosa, gums and dental surfaces. A “dental surface” herein is a surface of a natural tooth or a hard surface of artificial dentition including a crown, cap, filling, bridge, denture, or dental implant, for example.

An oral care composition herein can comprise about 0.01-15.0 wt % (e.g., ˜0.1-10 wt % or ˜0.1-5.0 wt %, ˜0.1-2.0 wt %) of insoluble alpha-glucan as disclosed herein, for example. Insoluble alpha-glucan comprised in an oral care composition can sometimes be provided therein as a thickening agent and/or dispersion agent, which may be useful to impart a desired consistency and/or mouth feel to the composition. One or more other thickening or dispersion agents can also be provided in an oral care composition herein, such as a carboxyvinyl polymer, carrageenan (e.g., L-carrageenan), natural gum (e.g., karaya, xanthan, gum arabic, tragacanth), colloidal magnesium aluminum silicate, or colloidal silica, for example.

An oral care composition herein may be a toothpaste or other dentifrice, for example. Such compositions, as well as any other oral care composition herein, can additionally comprise, without limitation, one or more of an anticaries agent, antimicrobial or antibacterial agent, anticalculus or tartar control agent, surfactant, abrasive, pH-modifying agent, foam modulator, humectant, flavorant, sweetener, pigment/colorant, whitening agent, and/or other suitable components. Examples of oral care compositions to which insoluble alpha-glucan can be added are disclosed in U.S. Patent Appl. Publ. Nos. 2006/0134025, 2002/0022006 and 2008/0057007, which are incorporated herein by reference.

An anticaries agent herein can be an orally acceptable source of fluoride ions. Suitable sources of fluoride ions include fluoride, monofluorophosphate and fluorosilicate salts as well as amine fluorides, including olaflur (N′-octadecyltrimethylendiamine-N,N,N′-tris(2-ethanol)-dihydrofluoride), for example. An anticaries agent can be present in an amount providing a total of about 100-20000 ppm, about 200-5000 ppm, or about 500-2500 ppm, fluoride ions to the composition, for example. In oral care compositions in which sodium fluoride is the sole source of fluoride ions, an amount of about 0.01-5.0 wt %, about 0.05-1.0 wt %, or about 0.1-0.5 wt %, sodium fluoride can be present in the composition, for example.

An antimicrobial or antibacterial agent suitable for use in an oral care composition herein includes, for example, phenolic compounds (e.g., 4-allylcatechol; p-hydroxybenzoic acid esters such as benzylparaben, butylparaben, ethylparaben, methylparaben and propylparaben; 2-benzylphenol; butylated hydroxyanisole; butylated hydroxytoluene; capsaicin; carvacrol; creosol; eugenol; guaiacol; halogenated bisphenolics such as hexachlorophene and bromochlorophene; 4-hexylresorcinol; 8-hydroxyquinoline and salts thereof; salicylic acid esters such as menthyl salicylate, methyl salicylate and phenyl salicylate; phenol; pyrocatechol; salicylanilide; thymol; halogenated diphenylether compounds such as triclosan and triclosan monophosphate), copper (II) compounds (e.g., copper (II) chloride, fluoride, sulfate and hydroxide), zinc ion sources (e.g., zinc acetate, citrate, gluconate, glycinate, oxide, and sulfate), phthalic acid and salts thereof (e.g., magnesium monopotassium phthalate), hexetidine, octenidine, sanguinarine, benzalkonium chloride, domiphen bromide, alkylpyridinium chlorides (e.g. cetylpyridinium chloride, tetradecylpyridinium chloride, N-tetradecyl-4-ethylpyridinium chloride), iodine, sulfonamides, bisbiguanides (e.g., alexidine, chlorhexidine, chlorhexidine digluconate), piperidino derivatives (e.g., delmopinol, octapinol), magnolia extract, grapeseed extract, rosemary extract, menthol, geraniol, citral, eucalyptol, antibiotics (e.g., augmentin, amoxicillin, tetracycline, doxycycline, minocycline, metronidazole, neomycin, kanamycin, clindamycin), and/or any antibacterial agents disclosed in U.S. Pat. No. 5,776,435, which is incorporated herein by reference. One or more antimicrobial agents can optionally be present at about 0.01-10 wt % (e.g., 0.1-3 wt %), for example, in the disclosed oral care composition.

An anticalculus or tartar control agent suitable for use in an oral care composition herein includes, for example, phosphates and polyphosphates (e.g., pyrophosphates), polyaminopropanesulfonic acid (AMPS), zinc citrate trihydrate, polypeptides (e.g., polyaspartic and polyglutamic acids), polyolefin sulfonates, polyolefin phosphates, diphosphonates (e.g., azacycloalkane-2,2-diphosphonates such as azacycloheptane-2,2-diphosphonic acid), N-methyl azacyclopentane-2,3-diphosphonic acid, ethane-1-hydroxy-1,1-diphosphonic acid (EHDP), ethane-1-amino-1,1-diphosphonate, and/or phosphonoalkane carboxylic acids and salts thereof (e.g., their alkali metal and ammonium salts). Useful inorganic phosphate and polyphosphate salts include, for example, monobasic, dibasic and tribasic sodium phosphates, sodium tripolyphosphate, tetrapolyphosphate, mono-, di-, tri- and tetra-sodium pyrophosphates, disodium dihydrogen pyrophosphate, sodium trimetaphosphate, sodium hexametaphosphate, or any of these in which sodium is replaced by potassium or ammonium. Other useful anticalculus agents in certain embodiments include anionic polycarboxylate polymers (e.g., polymers or copolymers of acrylic acid, methacrylic, and maleic anhydride such as polyvinyl methyl ether/maleic anhydride copolymers). Still other useful anticalculus agents include sequestering agents such as hydroxycarboxylic acids (e.g., citric, fumaric, malic, glutaric and oxalic acids and salts thereof) and aminopolycarboxylic acids (e.g., EDTA). One or more anticalculus or tartar control agents can optionally be present at about 0.01-50 wt % (e.g., about 0.05-25 wt % or about 0.1-15 wt %), for example, in the disclosed oral care composition.

A surfactant suitable for use in an oral care composition herein may be anionic, non-ionic, or amphoteric, for example. Suitable anionic surfactants include, without limitation, water-soluble salts of C₈₋₂₀ alkyl sulfates, sulfonated monoglycerides of C₈₋₂₀ fatty acids, sarcosinates, and taurates. Examples of anionic surfactants include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable non-ionic surfactants include, without limitation, poloxamers, polyoxyethylene sorbitan esters, fatty alcohol ethoxylates, alkylphenol ethoxylates, tertiary amine oxides, tertiary phosphine oxides, and dialkyl sulfoxides. Suitable amphoteric surfactants include, without limitation, derivatives of C₈₋₂₀ aliphatic secondary and tertiary amines having an anionic group such as a carboxylate, sulfate, sulfonate, phosphate or phosphonate. An example of a suitable amphoteric surfactant is cocoamidopropyl betaine. One or more surfactants are optionally present in a total amount of about 0.01-10 wt % (e.g., about 0.05-5.0 wt % or about 0.1-2.0 wt %), for example, in the disclosed oral care composition.

An abrasive suitable for use in an oral care composition herein may include, for example, silica (e.g., silica gel, hydrated silica, precipitated silica), alumina, insoluble phosphates, calcium carbonate, and resinous abrasives (e.g., a urea-formaldehyde condensation product). Examples of insoluble phosphates useful as abrasives herein are orthophosphates, polymetaphosphates and pyrophosphates, and include dicalcium orthophosphate dihydrate, calcium pyrophosphate, beta-calcium pyrophosphate, tricalcium phosphate, calcium polymetaphosphate and insoluble sodium polymetaphosphate. One or more abrasives are optionally present in a total amount of about 5-70 wt % (e.g., about 10-56 wt % or about 15-30 wt %), for example, in the disclosed oral care composition. The average particle size of an abrasive in certain embodiments is about 0.1-30 microns (e.g., about 1-20 microns or about 5-15 microns).

An oral care composition in certain embodiments may comprise at least one pH-modifying agent. Such agents may be selected to acidify, make more basic, or buffer the pH of a composition to a pH range of about 2-10 (e.g., pH ranging from about 2-8, 3-9, 4-8, 5-7, 6-10, or 7-9). Examples of pH-modifying agents useful herein include, without limitation, carboxylic, phosphoric and sulfonic acids; acid salts (e.g., monosodium citrate, disodium citrate, monosodium malate); alkali metal hydroxides (e.g. sodium hydroxide, carbonates such as sodium carbonate, bicarbonates, sesquicarbonates); borates; silicates; phosphates (e.g., monosodium phosphate, trisodium phosphate, pyrophosphate salts); and imidazole.

A foam modulator suitable for use in an oral care composition herein may be a polyethylene glycol (PEG), for example. High molecular weight PEGs are suitable, including those having an average molecular weight of about 200000-7000000 (e.g., about 500000-5000000 or about 1000000-2500000), for example. One or more PEGs are optionally present in a total amount of about 0.1-10 wt % (e.g. about 0.2-5.0 wt % or about 0.25-2.0 wt %), for example, in the disclosed oral care composition.

An oral care composition in certain embodiments may comprise at least one humectant. A humectant in certain embodiments may be a polyhydric alcohol such as glycerin, sorbitol, xylitol, or a low molecular weight PEG. Most suitable humectants also may function as a sweetener herein. One or more humectants are optionally present in a total amount of about 1.0-70 wt % (e.g., about 1.0-50 wt %, about 2-25 wt %, or about 5-15 wt %), for example, in the disclosed oral care composition.

A natural or artificial sweetener may optionally be comprised in an oral care composition herein. Examples of suitable sweeteners include dextrose, sucrose, maltose, dextrin, invert sugar, mannose, xylose, ribose, fructose, levulose, galactose, corn syrup (e.g., high fructose corn syrup or corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, and cyclamates. One or more sweeteners are optionally present in a total amount of about 0.005-5.0 wt %, for example, in the disclosed oral care composition.

A natural or artificial flavorant may optionally be comprised in an oral care composition herein. Examples of suitable flavorants include vanillin; sage; marjoram; parsley oil; spearmint oil; cinnamon oil; oil of wintergreen (methylsalicylate); peppermint oil; clove oil; bay oil; anise oil; eucalyptus oil; citrus oils; fruit oils; essences such as those derived from lemon, orange, lime, grapefruit, apricot, banana, grape, apple, strawberry, cherry, or pineapple; bean- and nut-derived flavors such as coffee, cocoa, cola, peanut, or almond; and adsorbed and encapsulated flavorants. Also encompassed within flavorants herein are ingredients that provide fragrance and/or other sensory effect in the mouth, including cooling or warming effects. Such ingredients include, without limitation, menthol, menthyl acetate, menthyl lactate, camphor, eucalyptus oil, eucalyptol, anethole, eugenol, cassia, oxanone, Irisone®, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), and menthone glycerol acetal (MGA). One or more flavorants are optionally present in a total amount of about 0.01-5.0 wt % (e.g., about 0.1-2.5 wt %), for example, in the disclosed oral care composition.

An oral care composition in certain embodiments may comprise at least one bicarbonate salt. Any orally acceptable bicarbonate can be used, including alkali metal bicarbonates such as sodium or potassium bicarbonate, and ammonium bicarbonate, for example. One or more bicarbonate salts are optionally present in a total amount of about 0.1-50 wt % (e.g., about 1-20 wt %), for example, in the disclosed oral care composition.

An oral care composition in certain embodiments may comprise at least one whitening agent and/or colorant. A suitable whitening agent is a peroxide compound such as any of those disclosed in U.S. Pat. No. 8,540,971, which is incorporated herein by reference. Suitable colorants herein include pigments, dyes, lakes and agents imparting a particular luster or reflectivity such as pearling agents, for example. Specific examples of colorants useful herein include talc; mica; magnesium carbonate; calcium carbonate; magnesium silicate; magnesium aluminum silicate; silica; titanium dioxide; zinc oxide; red, yellow, brown and black iron oxides; ferric ammonium ferrocyanide; manganese violet; ultramarine; titaniated mica; and bismuth oxychloride. One or more colorants are optionally present in a total amount of about 0.001-20 wt % (e.g., about 0.01-10 wt % or about 0.1-5.0 wt %), for example, in the disclosed oral care composition.

Additional components that can optionally be included in an oral composition herein include one or more enzymes (above), vitamins, and anti-adhesion agents, for example. Examples of vitamins useful herein include vitamin C, vitamin E, vitamin B5, and folic acid. Examples of suitable anti-adhesion agents include solbrol, ficin, and quorum-sensing inhibitors.

The present disclosure also concerns a method of treating a material. This method comprises contacting a material with an aqueous dispersion comprising insoluble alpha-glucan herein. Examples of an aqueous dispersion suitable for use in this method are described herein.

A material contacted with an aqueous dispersion in a contacting method herein can comprise a fabric in some aspects. A fabric herein can comprise natural fibers, synthetic fibers, semi-synthetic fibers, or any combination thereof. A semi-synthetic fiber herein is produced using naturally occurring material that has been chemically derivatized, an example of which is rayon. Non-limiting examples of fabric types herein include fabrics made of (i) cellulosic fibers such as cotton (e.g., broadcloth, canvas, chambray, chenille, chintz, corduroy, cretonne, damask, denim, flannel, gingham, jacquard, knit, matelassé, oxford, percale, poplin, plisse, sateen, seersucker, sheers, terry cloth, twill, velvet), rayon (e.g., viscose, modal, lyocell), linen, and Tencel®; (ii) proteinaceous fibers such as silk, wool and related mammalian fibers; (iii) synthetic fibers such as polyester, acrylic, nylon, and the like; (iv) long vegetable fibers from jute, flax, ramie, coir, kapok, sisal, henequen, abaca, hemp and sunn; and (v) any combination of a fabric of (i)-(iv). Fabric comprising a combination of fiber types (e.g., natural and synthetic) include those with both a cotton fiber and polyester, for example. Materials/articles containing one or more fabrics herein include, for example, clothing, curtains, drapes, upholstery, carpeting, bed linens, bath linens, tablecloths, sleeping bags, tents, car interiors, etc. Other materials comprising natural and/or synthetic fibers include, for example, non-woven fabrics, paddings, paper, and foams.

An aqueous dispersion that is contacted with a fabric can be, for example, a fabric care composition (e.g., laundry detergent, fabric softener). Examples of such compositions are described above. Thus, a treatment method in certain embodiments can be considered a fabric care method or laundry method if employing a fabric care composition therein. A fabric care composition herein is contemplated to effect one or more of the following fabric care benefits (i.e., surface substantive effects): wrinkle removal, wrinkle reduction, wrinkle resistance, fabric wear reduction, fabric wear resistance, fabric pilling reduction, extended fabric life, fabric color maintenance, fabric color fading reduction, reduced dye transfer, fabric color restoration, fabric soiling reduction, fabric soil release, fabric shape retention, fabric smoothness enhancement, anti-redeposition of soil on fabric, anti-greying of laundry, improved fabric hand/handle, and/or fabric shrinkage reduction.

Examples of conditions (e.g., time, temperature, wash/rinse volumes) for conducting a fabric care method or laundry method herein are disclosed in WO1997/003161 and U.S. Pat. Nos. 4,794,661, 4,580,421 and 5,945,394, which are incorporated herein by reference. In other examples, a material comprising fabric can be contacted with an aqueous dispersion herein: (i) for at least about 5, 10, 20, 30, 40, 50, 60, 70, 80, 90, 100, 110, or 120 minutes; (ii) at a temperature of at least about 10, 15, 20, 25, 30, 35, 40, 45, 50, 55, 60, 65, 70, 75, 80, 85, 90, or 95° C. (e.g., for laundry wash or rinse: a “cold” temperature of about 15-30° C., a “warm” temperature of about 30-50° C., a “hot” temperature of about 50-95° C.), (iii) at a pH of about 2, 3, 4, 5, 6, 7, 8, 9, 10, 11, or 12 (e.g., pH range of about 2-12, or about 3-11); (iv) at a salt (e.g., NaCl) concentration of at least about 0.5, 1.0, 1.5, 2.0, 2.5, 3.0, 3.5, or 4.0 wt %; or any combination of (i)-(iv).

The contacting step in a fabric care method or laundry method can comprise any of washing, soaking, and/or rinsing steps, for example. Contacting a material or fabric in still further embodiments can be performed by any means known in the art, such as mixing, shaking, spraying, treating, immersing, flushing, pouring on or in, combining, painting, coating, applying, and/or communicating an effective amount of insoluble alpha-glucan dispersion herein with the fabric or material. In still further embodiments, contacting may be used to treat a fabric to provide a surface substantive effect. As used herein, the term “fabric hand” or “handle” refers to a person's tactile sensory response towards fabric which may be physical, physiological, psychological, social or any combination thereof. In one embodiment, the fabric hand may be measured using a PhabrOmeter® System for measuring relative hand value (available from Nu Cybertek, Inc. Davis, Calif.) (American Association of Textile Chemists and Colorists [AATCC test method “202-2012, Relative Hand Value of Textiles: Instrumental Method”]).

In some aspects of treating a material comprising fabric, insoluble alpha-glucan of the aqueous dispersion adsorbs to the fabric. This feature is believed to render insoluble alpha-glucan herein useful as an anti-redeposition agent and/or anti-greying agent in fabric care compositions (in addition to its viscosity-modifying effect). An anti-redeposition agent or anti-greying agent herein helps keep soil from redepositing onto clothing in wash water after the soil has been removed. It is further contemplated that adsorption of insoluble alpha-glucan herein to a fabric enhances mechanical properties of the fabric.

Adsorption of insoluble alpha-glucan to a fabric herein can be measured using a colorimetric technique (e.g., Dubois et al., 1956, Anal. Chem. 28:350-356; Zemljič et al., 2006, Lenzinger Berichte 85:68-76; both incorporated herein by reference), for example, or any other method known in the art.

Other materials that can be contacted in the above treatment method include surfaces that can be treated with a dish detergent (e.g., automatic dishwashing detergent or hand dish detergent). Examples of such materials include surfaces of dishes, glasses, pots, pans, baking dishes, utensils and flatware made from ceramic material, china, metal, glass, plastic (e.g., polyethylene, polypropylene, polystyrene, etc.) and wood (collectively referred to herein as “tableware”). Thus, the treatment method in certain embodiments can be considered a dishwashing method or tableware washing method, for example. Examples of conditions (e.g., time, temperature, wash volume) for conducting a dishwashing or tableware washing method herein are disclosed in U.S. Pat. No. 8,575,083, which is incorporated herein by reference. In other examples, a tableware article can be contacted with an aqueous composition herein under a suitable set of conditions such as any of those disclosed above with regard to contacting a fabric-comprising material.

Other materials that can be contacted in the above treatment method include oral surfaces such as any soft or hard surface within the oral cavity including surfaces of the tongue, hard and soft palate, buccal mucosa, gums and dental surfaces (e.g., natural tooth or a hard surface of artificial dentition such as a crown, cap, filling, bridge, denture, or dental implant). Thus, a treatment method in certain embodiments can be considered an oral care method or dental care method, for example. Conditions (e.g., time, temperature) for contacting an oral surface with an aqueous composition herein should be suitable for the intended purpose of making such contact. Other surfaces that can be contacted in a treatment method also include a surface of the integumentary system such as skin, hair or nails.

Thus, certain embodiments of the present disclosure concern material (e.g., fabric) that comprises insoluble alpha-glucan herein. Such material can be produced following a material treatment method as disclosed herein, for example. A material may comprise insoluble alpha-glucan in some aspects if the compound is adsorbed to, or otherwise in contact with, the surface of the material.

Some aspects of a method of treating a material herein further comprise a drying step, in which a material is dried after being contacted with the aqueous dispersion. A drying step can be performed directly after the contacting step, or following one or more additional steps that might follow the contacting step (e.g., drying of a fabric after being rinsed, in water for example, following a wash in an aqueous dispersion herein). Drying can be performed by any of several means known in the art, such as air drying (e.g., ˜20-25° C.), or at a temperature of at least about 30, 40, 50, 60, 70, 80, 90, 100, 120, 140, 160, 170, 175, 180, or 200° C., for example. A material that has been dried herein typically has less than 3, 2, 1, 0.5, or 0.1 wt % water comprised therein. Fabric is a preferred material for conducting an optional drying step.

An aqueous dispersion comprising insoluble alpha-glucan herein can be used to prepare a film or coating, for example. In some aspects, a film or coating can be produced in a method comprising at least applying an aqueous dispersion as presently disclosed to a surface. In general, such a method can further comprise drying (completely or partially) the aqueous dispersion after it has been applied to the surface.

A film or coating can be a dried film or coating in some aspects, comprising less than about 3, 2, 1, 0.5, or 0.1 wt % water, for example. In some aspects, a film or coating can comprise about 20-40, 20-35, 20-30, 25-40, 25-35, or 25-30 wt % insoluble alpha-glucan, where the balance of material optionally is water, an aqueous solution, and/or a plasticizer. The amount of insoluble alpha-glucan comprised in a film or coating herein can be about, or at least about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, or 99.9 wt %, for example.

A film or coating herein can have a thickness of about, at least about, or up to about, 0.5, 0.6, 0.7, 0.8, 0.9, 1.0, 1.1, 1.2, 1.3, 1.4, 1.5, 2, 2.5, 5, 7.5, 10, 15.5, 15, 17.5, 20, 22.5, 25, 30, 35, 40, 45, 50, 75, 100, 150, 200, 0.5-1.5, 0.8-1.5, 1.0-1.5, 0.5-1.4, 0.8-1.4, or 1.0-1.4 mil (1 mil=0.001 inch), for instance. In some aspects, such thickness is uniform, which can be characterized by having a contiguous area that (i) is at least 20%, 30%, 40%, or 50% of the total film/coating area, and (ii) has a standard deviation of thickness of less than about 0.06, 0.05, or 0.04 mil. A film or coating herein can be characterized as thin (e.g., <2 mil) in some aspects. A film herein is typically a cast film.

A film or coating herein can optionally further comprise a plasticizer such as glycerol, propylene glycol, ethylene glycol, and/or polyethylene glycol. In some aspects, other film components (in addition to insoluble alpha-glucan herein) can be as disclosed in U.S. Patent. Appl. Publ. No. 2011/0151224, 2015/0191550, or 20190153674, U.S. Pat. No. 9,688,035 or 3,345,200, or International Patent Appl. Publ. No. WO2018/200437, all of which are incorporated herein by reference.

Also disclosed are articles comprising an adhesive, film, coating, or binder comprising insoluble alpha-glucan herein in a dry form, as produced using an aqueous dispersion herein, accordingly. Such articles (optionally, “coated articles”) comprise a substrate having at least one surface on which is disposed/deposited the coating, adhesive, film, or binder, in a substantially continuous or discontinuous manner. In some aspects, an article comprises paper, leather, wood, metal, polymer, fibrous material, masonry, drywall, plaster, and/or an architectural surface. An “architectural surface” herein is an external or internal surface of a building or other man-made structure. In some aspects, an article comprises a porous substrate such as in paper, cardboard, paperboard, corrugated board, a cellulosic substrate, a textile, or leather. Yet, in some aspects, an article can comprise a polymer such as polyamide, polyolefin, polylactic acid, polyethylene terephthalate (PET), poly(trimethylene terephthalate) (PTT), aramid, polyethylene sulfide (PES), polyphenylene sulfide (PPS), polyimide (PI), polyethylene imine (PEI), polyethylene naphthalate (PEN), polysulfone (PS), polyether ether ketone (PEEK), polyethylene, polypropylene, poly(cyclic olefins), poly(cyclohexylene dimethylene terephthalate), poly(trimethylene furandicarboxylate) (PTF), or cellophane. In some aspects, an article comprising a fibrous substrate is a fiber, yarn, fabric, fabric blend, textile, nonwoven, paper, or carpet. A fibrous substrate can contain natural and/or synthetic fibers, such as cotton, cellulose, wool, silk, rayon, nylon, aramid, acetate, polyurethane urea, acrylic, jute, sisal, sea grass, coir, polyamide, polyester, polyolefin, polyacrylonitrile, polypropylene, polyaramid, or blends thereof.

A film or coating herein can have grease/oil and/or oxygen barrier properties in some aspects. Such a film or coating can comprise, along with insoluble alpha-glucan herein, one or more components as disclosed in U.S. Patent. Appl. Publ. No. 20190153674 or International Patent Appl. Publ. No. WO2018/200437, which are each incorporated herein by reference. For example, a film or coating herein can comprise, optionally as a binder, one or more of polyvinyl alcohol, polyvinyl acetate, partially saponified polyvinyl acetate, silanol-modified polyvinyl alcohol, polyurethane, starch, corn dextrin, carboxymethyl cellulose, cellulose ethers, hydroxyethyl cellulose, hydroxypropyl cellulose, ethyl hydroxyethyl cellulose, methyl cellulose, alginates, sodium alginate, xanthan, carrageenan, casein, soy protein, guar gums, synthetic polymers, styrene butadiene latex, and/or styrene acrylate latex. A composition for preparing a film or coating in some aspects can comprise about 65, 70, 75, 80, 85, 65-85, 65-80, 70-85, or 70-80 wt % of a binder such as polyvinyl alcohol, and about 35, 30, 25, 20, 15, 15-35, 20-35, 15-30, or 20-30 wt % insoluble alpha-glucan as presently disclosed. In some aspects, a film or coating does not comprise starch, while in other aspects such as an oxygen barrier, starch can be included (e.g., as disclosed in U.S. Patent Appl. Publ. No. 2011/0135912 or U.S. Pat. No. 5,621,026 or 6,692,801, which are incorporated herein by reference). Grease/oil barrier properties of a coating composition herein can be evaluated using a standard “KIT” type test following Technical Association of the Pulp and Paper Industry (TAPPI) Test Method T-559 cm-02 (Grease resistance test for paper and paperboard, TAPPI Press, Atlanta, Ga., USA; incorporated herein by reference), for example. Good grease/oil barrier/resistance function is indicated in this test by values closer to 12 on a scale of 1 to 12. Oxygen barrier properties of a coating composition herein can be evaluated by measuring the oxygen transmission rate (OTR) of the coating; OTR can be determined, for example, according to ASTM F-1927-07 (2007, Standard Test Method for Determination of Oxygen Gas Transmission Rate, Permeability and Permeance at Controlled Relative Humidity Through Barrier Materials Using a Coulometric Detector, ASTM International, West Conshohocken, Pa.), which is incorporated herein by reference. OTR can be determined under relative humidity conditions of about 50%-80%, for example. Examples of substrates herein that can take advantage of a grease/oil and/or oxygen barrier coating include any of the forgoing substrates/surfaces, including a substrate comprising cellulose (e.g., paper, paperboard, cardboard, corrugated board, textile), polyethylene, polypropylene, poly lactic acid, poly(ethylene terephthalate) (e.g., MYLAR), poly(trimethylene terephthalate), polyamide, or poly(trimethylene furandicarboxylate).

A film or coating in some aspects can be in the form of an edible film or coating. Such a material can, in some aspects, comprise insoluble alpha-glucan herein and one or more components as described in U.S. Pat. Nos. 4,710,228, 4,543,370, 4,820,533, 4,981,707, 5,470,581, 5,997,918, 8,206,765, or 8999413, or U.S. Patent Appl. Publ. No. 2005/0214414, which are incorporated herein by reference. In some aspects, insoluble alpha-glucan replaces starch and/or starch derivatives in an edible film or coating, optionally as disclosed in any of the foregoing references. An edible film or coating can be on potato products (e.g., potato strips such as French fries), other vegetable products (e.g., zucchini, sweet potatoes, onions, okra, peppers, string beans), and mushrooms, for example. These and other food products having an edible film or coating herein can be fried or baked in some aspects, and/or the film or coating provides tenderness, moisture retention, crispness, and/or dietary fiber (in place of digestible starch).

A coating composition in some aspects, which can be used to prepare a coating herein, can comprise any of the foregoing components/ingredients/formulations. In some aspects, a coating composition is a latex composition, such as described below.

A aqueous dispersion comprising insoluble alpha-glucan herein can be a latex composition, or used to produce a latex composition. Examples of latex compositions herein include paint (e.g., primer, finishing/decorative), adhesives, coatings, and binders. Formulations and/or components (in addition to insoluble alpha-glucan herein) of a latex composition herein can be as described in, for example, U.S. Pat. Nos. 6,881,782, 3,440,199, 3,294,709, 5,312,863, 4,069,186 and 6,297,296, and International Patent Appl. Publ. No. WO2019046123, which are all incorporated herein by reference.

Insoluble alpha-glucan as presently disclosed can be present in a latex composition in any useful amount, such as at about, or at least about, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 40%, 45%, 50%, 55%, 60%, 65%, 70%, 75%, 0.01%-75% 0.01%-5%, 5%-20%, 20%-50%, or 50%-75% based on the weight of all the dispersed polymer solids of the latex.

A latex composition in some aspects can comprise a polymer polymerized from at least one ethylenically unsaturated monomer (e.g., monoethylenically unsaturated monomer); polyurethane; epoxy, and/or a rubber elastomer. Examples of monoethylenically unsaturated monomers herein include vinyl monomers, acrylic monomers, allylic monomers, acrylamide monomers, monocarboxylic unsaturated acids and dicarboxylic unsaturated acids.

Examples of suitable vinyl monomers of a polymer in a latex composition herein include any compounds having vinyl functionality (i.e., ethylenic unsaturation) such as vinyl esters (e.g., vinyl acetate, vinyl propionate, vinyl laurate, vinyl pivalate, vinyl nonanoate, vinyl decanoate, vinyl neodecanoate, vinyl butyrates, vinyl benzoates, vinyl isopropyl acetates), vinyl aromatic hydrocarbons (e.g., styrene, methyl styrenes and similar lower alkyl styrenes, chlorostyrene, vinyl toluene, vinyl naphthalene, divinyl benzene), vinyl aliphatic hydrocarbons (e.g., vinyl chloride; vinylidene chloride; alpha olefins such as ethylene, propylene and isobutylene; conjugated dienes such as 1,3-butadiene, methyl-2-butadiene, 1,3-piperylene, 2,3-dimethyl butadiene, isoprene, cyclohexene, cyclopentadiene, and dicyclopentadiene) and vinyl alkyl ethers (e.g., methyl vinyl ether, isopropyl vinyl ether, n-butyl vinyl ether, isobutyl vinyl ether), but excluding compounds having acrylic functionality (e.g., acrylic acid, methacrylic acid, esters of such acids, acrylonitrile, acrylamides). In some aspects, a latex composition herein comprises a vinyl acetate-ethylene copolymer, carboxylated vinyl acetate-ethylene copolymer, and/or or polyvinyl acetate.

Examples of suitable acrylic monomers of a polymer in a latex composition herein include alkyl acrylates, alkyl methacrylates, acrylate acids, methacrylate acids, aromatic derivatives of acrylic and methacrylic acid, acrylamides, and acrylonitrile. Typically, alkyl acrylate and methacrylic monomers (also referred to as alkyl esters of acrylic or methacrylic acid) have an alkyl ester portion containing from 1 to about 18 carbon atoms per molecule, or from 1 to about 8 carbon atoms per molecule. Suitable acrylic monomers include, for example, methyl acrylate and methacrylate, ethyl acrylate and methacrylate, butyl acrylate and methacrylate, propyl acrylate and methacrylate, 2-ethyl hexyl acrylate and methacrylate, cyclohexyl acrylate and methacrylate, decyl acrylate and methacrylate, isodecyl acrylate and methacrylate, benzyl acrylate and methacrylate, isobornyl acrylate and methacrylate, neopentyl acrylate and methacrylate, and 1-adamantyl methacrylate. If acid functionality is desired, acids such as acrylic acid or methacrylic acid can also be used.

A latex composition in some aspects comprises a polyurethane polymer. Examples of suitable polyurethane polymers are those comprising polysaccharides as disclosed in International Patent Appl. Publ. No. WO2018/017789, which is incorporated herein by reference. A latex comprising a polyurethane can be prepared, for example, as disclosed in U.S. Patent Appl. Publ. No. 2016/0347978, which is incorporated herein by reference, and/or comprise the reaction product of one or more polyisocyanates with one or more polyols. Useful polyols include polycarbonate polyols, polyester polyols and polyether polyols, for example. Polycarbonate polyurethane herein can be formed as the reaction product of a polyol such as 1,3-propanediol, 1,4-butanediol, 1,6-hexanediol, diethylene glycol, or tetraethylene glycol, with a diaryl carbonate such as diphenyl carbonate or phosgene. At least one polyisocyanate herein can be an aliphatic polyisocyanate, aromatic polyisocyanate, or polyisocyanate that has both aromatic and aliphatic groups. Examples of polyisocyanates include 1,6-hexamethylene diisocyanate, isophorone diisocyanate, 2,4-toluene diisocyanate, 2,6-toluene diisocyanate, mixtures of 2,4- and 2,6-toluene diisocyanate, bis(4-isocyanatocyclohexyl) methane, 1,3-bis(1-isocyanato-1-methylethyl)benzene, bis(4-isocyanatophenyl)methane, 2,4′-diphenylmethane diisocyanate, 2,2′-diphenylmethane diisocyanate, 2,4-diisocyanatotoluene, bis(3-isocyanatophenyl)methane, 1,4-diisocyanatobenzene, 1,3-diisocyanato-o-xylene, 1,3-diisocyanato-p-xylene, 1,3-diisocyanato-m-xylene, 2,4-diisocyanato-1-chlorobenzene, 2,4-diisocyanato-1-nitrobenzene, 2,5-diisocyanato-1-nitrobenzene, m-phenylene diisocyanate, hexahydrotoluene diisocyanate, 1,5-naphthalene diisocyanate, 1-methoxy-2,4-phenylene diisocyanate, 4,4′-biphenylmethane diisocyanate, 4,4′-biphenylene diisocyanate, 3,3′-dimethyl-4,4′-diphenylmethane, diisocyanate, 3,3′-4,4′-diphenylmethane diisocyanate, and 3,3′-dimethyldiphenylmethane-4,4′-diisocyanate. Also useful herein are polyisocyanate homopolymers comprising allophanate, biuret, isocyanurate, iminooxadiazinedione, or carbodiimide groups, for example. A polyol herein can be any polyol comprising two or more hydroxyl groups, for example, a C2 to C12 alkane diol, ethylene glycol, 1,2-propylene glycol, 1,3-propylene glycol, isomers of butane diol, pentane diol, hexane diol, heptane diol, octane diol, nonane diol, decane diol, undecane diol, dodecane diol, 2-methyl-1,3-propane diol, 2,2-dimethyl-1,3-propane diol (neopentyl glycol), 1,4-bis(hydroxymethyl)cyclohexane, 1,2,3-propane triol (glycerol), 2-hydroxymethyl-2-methyl-1,3-propanol (trimethylolethane), 2-ethyl-2-hydroxymethyl-1,3-propanediol (trimethylolpropane), 2,2-bis(hydroxymethyl)-1,3-propane diol (pentaerythritol); 1,4,6-octanetriol, chloropentanediol; glycerol monoalkyl ether; glycerol monoethyl ether; diethylene glycol; 1,3,6-hexanetriol; 2-methylpropanediol; 2,2,4-trimethyl-1,3-pentanediol, cyclohexanedimethanol, polymeric polyols, for example, polyether polyols or polyester polyols. In some aspects, a polyol herein can be poly(oxytetramethylene) glycol, polyethylene glycol, or poly 1,3-propane diol. A polyol in some aspects can be polyester polyol, such as one produced by transesterification of aliphatic diacids with aliphatic diols. Suitable aliphatic diacids include, for example, C3 to C10 diacids, malonic acid, succinic acid, glutaric acid, adipic acid, pimelic acid, suberic acid, azelic acid, sebacic acid. In some aspects, aromatic and/or unsaturated diacids can be used to form a polyester polyol.

A latex composition in some aspects comprises an epoxy polymer/resin (polyepoxide), such as bisphenol A epoxy resin, bisphenol F epoxy resin, Novolac epoxy resin, aliphatic epoxy resin, or glycidylamine epoxy resin.

A latex composition in some aspects comprises a rubber elastomer. In some aspects, a rubber elastomer can include one or more diene-based sulfur-vulcanizable elastomers having a glass transition temperature (Tg) below −30° C., as determined, for example, by dynamic mechanical analysis. In further examples, a rubber elastomer herein includes natural rubber, synthetic polyisoprene, polybutadiene rubber, styrene/butadiene copolymer rubber, ethylene propylene diene monomer rubber, hydrogenated nitrile butadiene rubber, neoprene, styrene/isoprene/butadiene terpolymer rubber, butadiene/acrylonitrile rubber, polyisoprene rubber, isoprene/butadiene copolymer rubber, nitrile rubber, ethylene-acrylic rubber, butyl and halobutyl rubber, chlorosulfonated polyethylene, fluoroelastomer, hydrocarbon rubber, polybutadiene, and silicone rubber.

A latex composition herein comprises insoluble alpha-glucan dispersed in a dispersion (other polymers such as above can optionally be dispersed along with the alpha-glucan) or emulsion, where the liquid component of the latex can be water or an aqueous solution. An aqueous solution of a latex in some aspects can comprise an organic solvent that is either miscible or immiscible with water. Suitable organic solvents herein include acetone, methyl ethyl ketone, butyl acetate, tetrahydrofuran, methanol, ethanol, isopropanol, diethyl ether, glycerol ethers, hexane, toluene, dimethyl acetamide, dimethylformamide, and dimethyl sulfoxide.

A latex composition herein can further comprise one or more additives in some aspects. Examples of additives herein include dispersants, rheological aids, antifoams, foaming agents, adhesion promoters, flame retardants, bactericides, fungicides, preservatives, optical brighteners, fillers, anti-settling agents, coalescing agents, humectants, buffers, pigments/colorants (e.g., metallic oxides, synthetic organic pigments, carbon black), viscosity modifiers, antifreeze, surfactants, binders, crosslinking agents, anticorrosion agents, hardeners, pH regulators, salts, thickeners, plasticizers, stabilizers, extenders, and matting agents. Examples of pigments herein include titanium dioxide (TiO₂), calcium carbonate, diatomaceous earth, mica, hydrated aluminum oxide, barium sulfate, calcium silicate, clay, silica, talc, zinc oxide, aluminum silicate, nepheline syenite, and mixtures thereof. In some aspects, a latex composition is essentially free from (e.g., less than 1, 0.5, 0.1, or 0.01 wt % of component) starch, starch derivative (e.g., hydroxyalkyl starch), cellulose, and/or cellulose derivative (e.g., carboxymethyl cellulose).

A latex composition in the form of a paint or other coloring agent herein can have a pigment volume concentration (PVC) of about 3% to about 80% in some aspects. As examples, a flat paint can have a PVC in the range of about 55-80%, a primer or undercoat can have a PVC in the range of about 30-50%, and/or a gloss colored paint can have a PVC in the range of about 3-20%. A paint or other coloring agent in some aspects can have a PVC of about 55%, 60%, 65%, 70%, 75%, 80%, 55-80%, 55-75%, 55-70%, 60-80%, 60-75%, 60-70%, 63-67%, 64-66%, 65-80%, 65-75%, or 65-70%. A PVC value herein can be that of a particular pigment (or mix of pigments) such as those disclosed above (e.g., titanium dioxide), for instance. Insoluble alpha-glucan of the present disclosure is believed to provide one or more physical properties to a latex composition (e.g., for use as a paint or other coloring agent): increase opacity, less pigment needed, increased hardness, reduced tackiness, decreased gloss (i.e., providing a matte effect), increased shear strength, better abrasion resistance, improved dry time, improved fade resistance, lower blistering, and/or improved hand (a less tacky feel), for example, as compared to a latex composition that only differs by not comprising the insoluble alpha-glucan.

A latex composition herein can be applied to the substrate of an article (above) using any method known in the art. Typically, after application of the latex composition, at least a portion of the aqueous solution is removed, for example by drying, to provide an adhesive, film, coating, or binder comprising the latex composition in a dry or semi-dry form. Suitable application methods include air knife coating, rod coating, bar coating, wire bar coating, spray coating, brush coating, cast coating, flexible blade coating, gravure coating, jet applicator coating, short dwell coating, slide hopper coating, curtain coating, flexographic coating, size-press coating, reverse roll coating, and transfer roll coating. A latex composition can be applied on at least a portion of a substrate, and can be in one or more coats/applications, for example.

Non-limiting examples of compositions and methods disclosed herein include:

1. A method of producing an aqueous dispersion, the method comprising: (a) providing a first composition comprising at least 88% insoluble alpha-glucan by weight of the first composition, wherein at least 50% of the glycosidic linkages of the insoluble alpha-glucan are alpha-1,3 glycosidic linkages, and (b) mixing at least aqueous liquid and the first composition to produce an aqueous dispersion having about 0.5% to about 10% by weight of the insoluble alpha-glucan, wherein the mixing comprises applying pressure of at least 1000 pounds per square inch (psi).
2-1. The method of embodiment 1, wherein the pressure of at least 1000 psi is applied by pressure homogenization.
2-2. The method of embodiment 1 or 2-1, wherein the mixing comprises applying pressure of at least 7000 pounds psi.
3. The method of embodiment 1, 2-1, or 2-2, wherein the first composition comprises at least 95% insoluble alpha-glucan by weight of the first composition.
4. The method of embodiment 1, 2-1, 2-2, or 3, wherein the aqueous dispersion has about 0.5% to about 4.5% by weight of the insoluble alpha-glucan.
5. The method of embodiment 1, 2-1, 2-2, 3, or 4, wherein at least 90% of the glycosidic linkages of the insoluble alpha-glucan are alpha-1,3 linkages.
6. The method of embodiment 1, 2-1, 2-2, 3, 4, or 5, wherein the weight average degree of polymerization (DPw) of the insoluble alpha-glucan is at least about 15 or 100.
7. The method of embodiment 1, 2-1, 2-2, 3, 4, 5, or 6, wherein at least 60% by weight of the insoluble alpha-glucan particles in the aqueous dispersion have a diameter of less than 30 micrometers.
8. The method of embodiment 1, 2-1, 2-2, 3, 4, 5, 6, or 7, wherein the insoluble alpha-glucan is dispersed through at least about 60% of the volume of the aqueous dispersion.
9. The method of embodiment 1, 2-1, 2-2, 3, 4, 5, 6, 7, or 8, wherein the insoluble alpha-glucan provided in the first composition was dried by agitated air drying.
10. The method of embodiment 1, 2-1, 2-2, 3, 4, 5, 6, 7, 8, or 9, wherein the insoluble alpha-glucan was in the form of a wet cake prior to being provided in the first composition, wherein the wet cake comprised about 10% to about 55% by weight of the insoluble alpha-glucan and about 45% to 90% by weight of an aqueous fluid.
11. The method of embodiment 1, 2-1, 2-2, 3, 4, 5, 6, 7, 8, 9, or 10, wherein the first composition comprises less than about 0.35% by weight of soluble sugars on a dry weight basis.
12. The method of embodiment 1, 2-1, 2-2, 3, 4, 5, 6, 7, 8, 9, 10, or 11, wherein the insoluble alpha-glucan provided in the first composition was produced in an enzymatic reaction comprising at least water, sucrose and a glucosyltransferase enzyme that synthesizes insoluble alpha-glucan at a yield of at least about 75%.
13. The method of embodiment 1, 2-1, 2-2, 3, 4, 5, 6, 7, 8, 9, 10, 11, or 12, wherein the insoluble alpha-glucan provided in the first composition was produced in an enzymatic reaction comprising at least water, sucrose, a glucosyltransferase enzyme that synthesizes insoluble alpha-glucan, and oligosaccharides that (i) comprise alpha-1,3 and alpha-1,6 glycosidic linkages, and/or (ii) are produced from a glucosyltransferase reaction, wherein the oligosaccharides were added during preparation of the enzymatic reaction.
14. The method of embodiment 1, 2-1, 2-2, 3, 4, 5, 6, 7, 8, 9, 10, 11, 12, or 13, wherein the aqueous dispersion has a viscosity that is at least about 50% higher than the viscosity that the aqueous dispersion would have had if it was instead prepared by mixing at an rpm (revolutions per minute) of no more than 10000.
15. An aqueous dispersion produced according to the method of embodiment 1, 2-1, 2-2, 3, 4, 5, 6, 7, 8, 9, 10, 11, 12, 13, or 14.
16. An aqueous dispersion comprising about 0.5 wt % to about 10 wt % insoluble alpha-glucan, wherein: at least 60% by weight of the insoluble alpha-glucan particles in the aqueous dispersion have a diameter of less than 30 micrometers, the insoluble alpha-glucan is dispersed through at least about 60% of the volume of the aqueous dispersion, and at least 50% of the glycosidic linkages of the insoluble alpha-glucan are alpha-1,3 glycosidic linkages, optionally wherein the aqueous dispersion is produced according to the method of embodiment 1, 2-1, 2-2, 3, 4, 5, 6, 7, 8, 9, 10, 11, 12, 13, or 14.
17. The aqueous dispersion of embodiment 15 or 16, wherein the aqueous dispersion is comprised in a household care product, personal care product, industrial product, pharmaceutical product, or food product.

Non-limiting examples of compositions and methods disclosed herein also include:

1a. A method of producing an aqueous dispersion, the method comprising: (a) providing a first composition comprising (i) about 10% to 55% insoluble alpha-glucan by weight of the first composition, and (ii) a balance of water or aqueous solution up to 100% by weight of the first composition, wherein at least 50% of the glycosidic linkages of the insoluble alpha-glucan are alpha-1,3 glycosidic linkages; and (b) mixing at least aqueous liquid and the first composition to produce an aqueous dispersion having about 0.01% to about 8.5% by weight of the insoluble alpha-glucan, wherein the mixing comprises applying pressure of at least 1000 pounds per square inch (psi).
2a-1. The method of embodiment 1a, wherein the pressure of at least 1000 psi is applied by pressure homogenization.
2a-2. The method of embodiment 1a or 2a-1, wherein the mixing comprises applying pressure of at least 7000 pounds psi.
3a. The method of embodiment 1a, 2a-1, or 2a-2, wherein the first composition comprises about 10% to 40% insoluble alpha-glucan by weight of the first composition.
4a. The method of embodiment 1a, 2a-1, 2a-2, or 3a, wherein the aqueous dispersion has about 0.5% to about 4.5% by weight of the insoluble alpha-glucan.
5a. The method of embodiment 1a, 2a-1, 2a-2, 3a, or 4a, wherein at least 90% of the glycosidic linkages of the insoluble alpha-glucan are alpha-1,3 linkages.
6a. The method of embodiment 1a, 2a-1, 2a-2, 3a, 4a, or 5a, wherein the weight average degree of polymerization (DPw) of the insoluble alpha-glucan is at least about 15 or 100.
7a. The method of embodiment 1a, 2a-1, 2a-2, 3a, 4a, 5a, or 6a, wherein at least 90% by weight of the insoluble alpha-glucan particles in the aqueous dispersion have a diameter of less than 30 micrometers.
8a. The method of embodiment 1a, 2a-1, 2a-2, 3a, 4a, 5a, or 6a, wherein at least 95% by weight of the insoluble alpha-glucan particles in the aqueous dispersion have a diameter of less than 50 micrometers.
9a. The method of embodiment 1a, 2a-1, 2a-2, 3a, 4a, 5a, 6a, 7a, or 8a, wherein the insoluble alpha-glucan is dispersed through at least about 80% of the volume of the aqueous dispersion.
10a. The method of embodiment 1a, 2a-1, 2a-2, 3a, 4a, 5a, 6a, 7a, 8a, or 9a, wherein the first composition comprises less than about 0.35% by weight of soluble sugars on a dry weight basis.
11a. The method of embodiment 1a, 2a-1, 2a-2, 3a, 4a, 5a, 6a, 7a, 8a, 9a, or 10a, wherein the insoluble alpha-glucan provided in the first composition was produced in an enzymatic reaction comprising at least water, sucrose and a glucosyltransferase enzyme that synthesizes insoluble alpha-glucan at a yield of at least about 75%.
12a. The method of embodiment 1a, 2a-1, 2a-2, 3a, 4a, 5a, 6a, 7a, 8a, 9a, 10a, or 11a, wherein the insoluble alpha-glucan provided in the first composition was produced in an enzymatic reaction comprising at least water, sucrose, a glucosyltransferase enzyme that synthesizes insoluble alpha-glucan, and oligosaccharides that (i) comprise alpha-1,3 and alpha-1,6 glycosidic linkages, and/or (ii) are produced from a glucosyltransferase reaction, wherein the oligosaccharides were added during preparation of the enzymatic reaction.
13a. The method of embodiment 1a, 2a-1, 2a-2, 3a, 4a, 5a, 6a, 7a, 8a, 9a, 10a, 11a, or 12a, wherein the aqueous dispersion has a viscosity that is at least about 200% higher than the viscosity that the aqueous dispersion would have had if it was instead prepared by mixing at an rpm (revolutions per minute) of no more than 10000.
14a. An aqueous dispersion produced according to the method of embodiment 1a, 2a-1, 2a-2, 3a, 4a, 5a, 6a, 7a, 8a, 9a, 10a, 11a, 12a, or 13a.
15a. An aqueous dispersion comprising about 0.01 wt % to about 8.5 wt % insoluble alpha-glucan, wherein: at least 90% by weight of the insoluble alpha-glucan particles in the aqueous dispersion have a diameter of less than 30 micrometers, the insoluble alpha-glucan is dispersed through at least about 80% of the volume of the aqueous dispersion, and at least 50% of the glycosidic linkages of the insoluble alpha-glucan are alpha-1,3 glycosidic linkages, optionally wherein the aqueous dispersion is produced according to the method of embodiment 1a, 2a-1, 2a-2, 3a, 4a, 5a, 6a, 7a, 8a, 9a, 10a, 11a, 12a, or 13a.
16a. The aqueous dispersion of embodiment 14a or 15a, wherein the aqueous dispersion is comprised in a household care product, personal care product, industrial product, pharmaceutical product, or food product.

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Example 1

### Preparing High Viscosity Aqueous Dispersions of Insoluble Alpha-Glucan (Dried or Never-Dried) Using a High Shear Disperser

This Example describes preparing high viscosity aqueous dispersions of insoluble alpha-glucan. In particular, aqueous dispersions were prepared with relatively low concentrations (˜4 wt %) of insoluble alpha-1,3-glucan using an inline high pressure homogenizer. In addition to being viscous, these dispersions were stable and contained particles of relatively homogeneous size. These features applied to dispersions of wet, never-dried alpha-1,3-glucan as well as to dispersions of dry alpha-1,3-glucan.

Insoluble alpha-1,3-glucan used in this Example was prepared in a manner similar to what is described in U.S. Patent Appl. Publ. Nos. 2018/0340199 and 2019/0078063, which are both incorporated herein by reference. In general, a glucan synthesis reaction was performed comprising water, sucrose, buffer, filtrate from an earlier glucan synthesis reaction (contains, e.g., gluco-oligosaccharide byproducts of the earlier glucan synthesis reaction), and an amino acid-modified, high product-yielding glucosyltransferase enzyme. Following the reaction, the alpha-1,3-glucan product (insoluble, ˜100% alpha-1,3 linkages, DPw of about 800) was filtered and washed to remove most fructose and other residual soluble sugars (e.g., glucose, sucrose, leucrose, DP2-DP8 gluco-oligosaccharides). Samples of the washed product were then either collected into wet cakes (never-dried) of about 10-40 wt % solids or dried in a rotary dryer to powders of about 88-95 wt % solids.

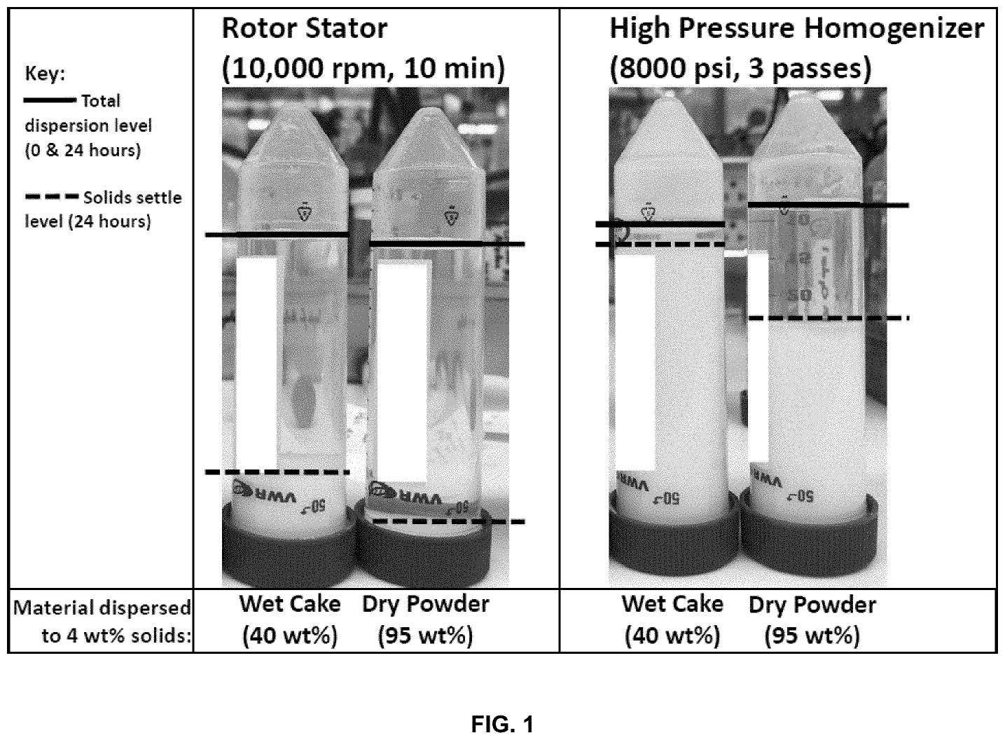

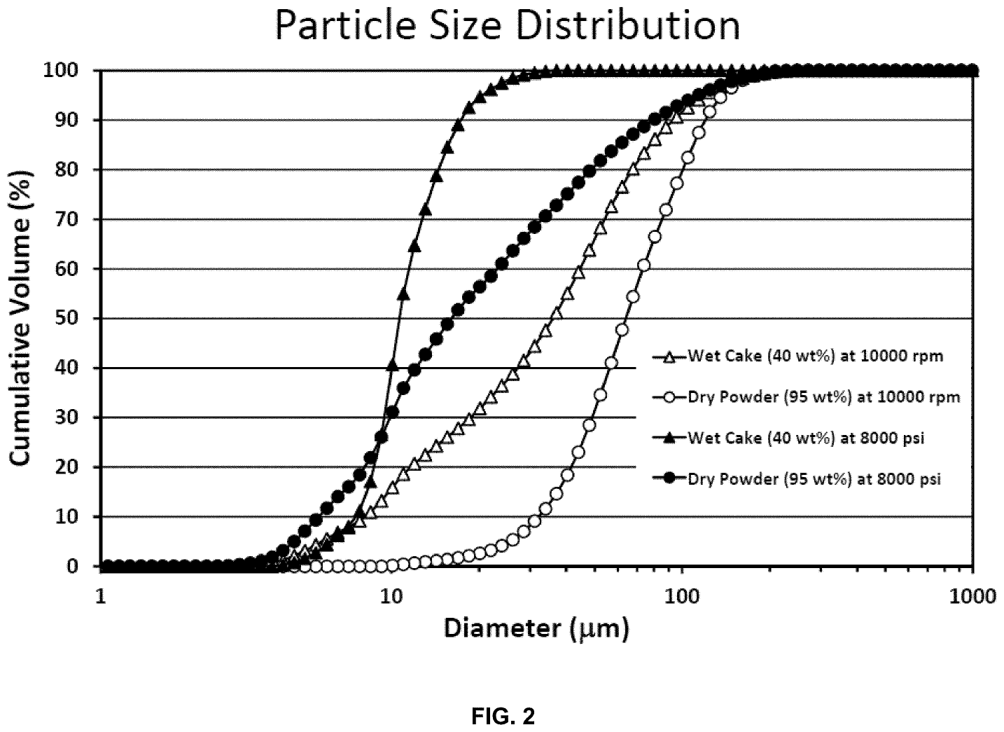

In this Example, 10 wt % and 40 wt % solids wet cakes and 95 wt % solids dry powder were each mixed with deionized water by hand-shaking (no dispersing device used) to 4 wt % solids. Samples of each of these preparations were then processed at room temperature with a hand-held rotor stator (IKA T-25) at 10000 rpm (revolutions per minute) for 10 minutes or three passes with a pressure homogenizer (APV Gaulin Inc., Wilmington, Mass.; model no. 15MER-8TBA, 15 gallons per hour capacity; 8000 psi capacity; 2-stage homogenizing valve assembly; single plunger, 2⅛″ stroke; tapered seal ball valve cylinder; driven by 3-hp, 60-Hz 230/460-volt 9.2/4.6-amp 3-phase 1730-rpm motor) at 5000 or 8000 psi. The resulting aqueous dispersions were then analyzed for viscosity (using a BROOKFIELD viscometer set at 100 rpm, Table 1 below), phase separation (by visual observation, FIG. 1), and particle size (by light scatter analysis, FIG. 2).

TABLE 1
Viscosity Measurements of Aqueous Dispersions of
Alpha-1,3-Glucan Wet Cake and Dry Powder Samples

| | Rotor Stator | High Pressure Homogenizer | |
|---|---|---|---|
| | 10000 rpm | 5000 psi | 8000 psi |
| Alpha-1,3-Glucan | 10 minutes | three passes | three passes |

| | | | | | | |
|---|---|---|---|---|---|---|
| Wet Cake | 24.8 | cps | 139.6 | cps | 141.6 | cps |
| 10 wt % solids^{a} | | | | | | |
| Wet Cake | 13 | cps | 83.6 | cps | 170.8 | cps |
| 40 wt % solids^{a} | | | | | | |
| Dry Powder | 13.6 | cps | 19.4 | cps | 44.4 | cps |
| 95 wt % solids^{a} | | | | | | |

^{a}Listed material is what was used to prepare an aqueous dispersion with 4 wt % insoluble alpha-1,3-glucan.

The data in Table 1 show that alpha-1,3-glucan dispersions prepared by high pressure homogenization have significantly higher viscosities as compared to the viscosities of dispersions prepared with a rotor stator. It is notable that the dispersion of dry alpha-1,3-glucan powder made by homogenization at 8000 psi had higher viscosity than the viscosities of dispersions of wet cake and dry powder samples made with the lower intensity disperser (rotor stator, 10000 rpm). This result, especially seeing a significant difference in viscosities of dispersions of dry alpha-1,3-glucan made under high shear (8000 psi) and less shear (rotor stator), was unexpected in view of U.S. Patent Appl. Publ. No. 2018/0273731, which discloses that dispersing dry alpha-1,3-glucan under high pressure did not result in any significant change in viscosity or homogeneity as compared to the viscosity of dry alpha-1,3-glucan dispersed under low shear conditions. It is estimated that, based on the data in Table 1, application of a high shear of 7000 psi (3 passes) in dispersing dry alpha-1,3-glucan would result in a dispersion with a viscosity of about 36 cps.

In addition to viscosity, dispersion stability and particle size can be used to evaluate the quality of alpha-1,3-glucan dispersions. FIG. 1 shows that alpha-1,3-glucan in both wet cake (40 wt % solids) and dry (95 wt % solids) forms, when dispersed at room temperature to 4 wt % in water under high shear (8000 psi), exhibited significantly enhanced stability as compared to the same materials as dispersed with less shear (rotor stator, 10000 rpm). Twenty-four hours after each set of dispersions were made, those made under 8000 psi exhibited far less particle settling compared to the dispersions made with less shear (FIG. 1). As with the above results over viscosity, the enhanced stability of dispersions of dry alpha-1,3-glucan made under high shear (8000 psi) (which also reflects enhanced homogeneity) was unexpected in view of the disclosure of U.S. Patent Appl. Publ. No. 2018/0273731. Alpha-1,3-glucan particles were dispersed through roughly 70% of the volume of the aqueous dispersion of dry alpha-1,3-glucan made by homogenization at 8000 psi (FIG. 1). In the aqueous dispersion made by homogenizing wet cake (40 wt % solids) at 8000 psi, alpha-1,3-glucan particles were dispersed through roughly 95% of the volume of the aqueous dispersion (FIG. 1).

FIG. 2 shows particle size distributions of the aqueous dispersions prepared above. Briefly, alpha-1,3-glucan in both wet cake (40 wt % solids) and dry (95 wt % solids) forms, when dispersed at room temperature to 4 wt % in water under high shear (8000 psi), exhibited significantly smaller particle sizes as compared to the same materials as dispersed with less shear (rotor stator, 10000 rpm). Particle size distributions were determined at room temperature with each dispersion upon its preparation by light scatter analysis. Roughly 66-68% of particles of aqueous dispersions of dry alpha-1,3-glucan made under high shear (8000 psi) had a diameter of less than 30 micrometers, whereas only about 7-9% of particles of the same material as dispersed under 10000 rpm had a diameter less than 30 micrometers (FIG. 2). Interestingly, while about 50% of particles of aqueous dispersions of dry alpha-1,3-glucan made under high shear (8000 psi) had a diameter of less than about 17 micrometers, only about 28% of particles of aqueous dispersions of wet alpha-1,3-glucan made with less shear (rotor stator, 10000 rpm) were less than this size (FIG. 2). FIG. 2 also shows that well over about 98% of alpha-1,3-glucan particles of aqueous dispersions of wet cake (40 wt % solids) made under high shear (8000 psi) had a diameter of less than about 30 micrometers, whereas only about 42% of particles of aqueous dispersions of wet alpha-1,3-glucan made with less shear (rotor stator, 10000 rpm) were less than this size. Also, about 100% of alpha-1,3-glucan particles of aqueous dispersions of wet cake (40 wt % solids) made under high shear (8000 psi) had a diameter of less than about 50 micrometers, whereas about 64% of particles of aqueous dispersions of wet alpha-1,3-glucan made with less shear (rotor stator, 10000 rpm) were less than this size (FIG. 2).

### Example 2

### High Viscosity Aqueous Dispersions of Never-Dried Insoluble Alpha-Glucan Prepared Using a Multiple Pass Procedure

This Example describes preparing a high viscosity aqueous dispersion of insoluble alpha-1,3-glucan using a low to medium intensity dispersing unit with multiple passes to achieve dispersion qualities of a dispersion produced using a high intensity dispersing unit.

The insoluble alpha-1,3-glucan used in this Example was identical to the alpha-1,3-glucan used in Example 1. Samples of a 10 wt % solids wet cake (never-dried) of alpha-1,3-glucan (as prepared in Example 1) were dispersed as described below.

Two different types of inline dispersers with low to medium intensity were selected: an inline rotor stator (IKA DR 2000/20) and an inline colloid mill (IKA MK 2000/20). The inline rotor stator and colloid mill are considered to be low to medium intensity dispersers relative to a pressure homogenizer. When the disperser size is fixed to similar flow rates, the pressure homogenizer requires 5-10 times more energy compared to an inline rotor stator or a colloid mill (Table 2).

TABLE 2
Comparison of Different Dispersers

| | Manufac- | | Flow Rate | Operating | |
|---|---|---|---|---|---|
| Disperser Type | turer | Model | (gph)^{a} | Power (HP)^{a} | |
| Inline Colloid Mill | IKA | MK 2000/20 | 3600 | 33 | HP |
| Inline Rotor Stator | IKA | DR 2000/20 | 2700 | 66 | HP |
| Pressure | APV | R315 60.175 | 3600 | 350 | HP |
| Homogenizer | Gaulin | | | | |

^{a}Flow rate and the operating power in this comparison are specific for dispersing 10 wt % insoluble alpha-1,3-glucan. gph (gallons per hour), HP (horsepower).

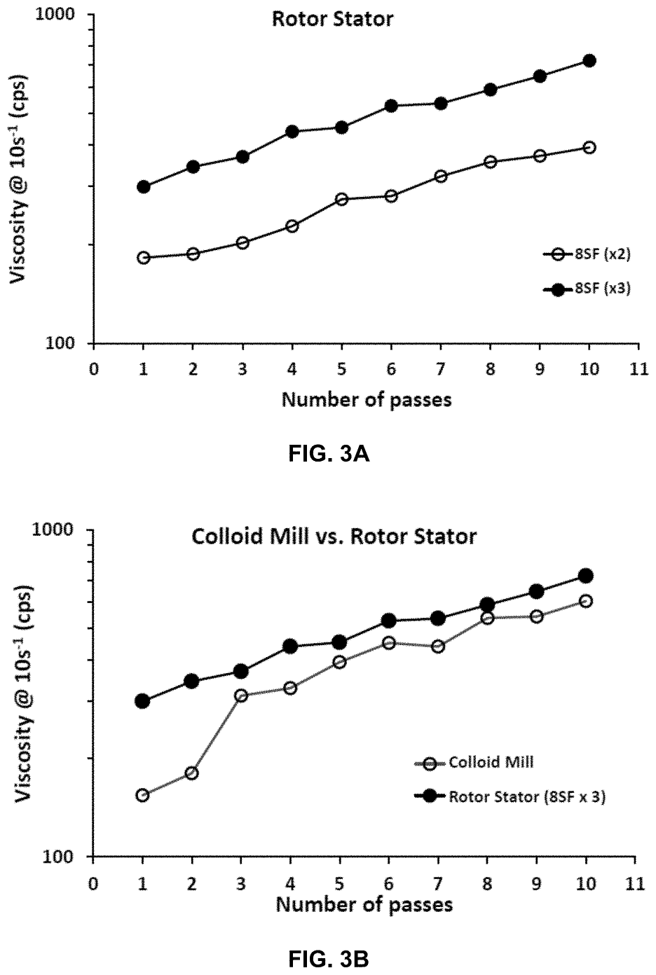

For the inline rotor stator (Table 2), two different operating formats were tested: (1) stack of two 8SF generators, and (2) stack of three 8SF generators. 8SF generators are represented by IKA to produce superfine dispersions; a stack of three of this generator type is the maximum stacking recommended for use in a single disperser. FIG. 3A shows the viscosities (measured under steady state shear of 10 s⁻¹) resulting from using multiple pass dispersal processing of alpha-1,3-glucan with the rotor stator as set up with the two different stacking schemes. For both stacking schemes, shear viscosity increased linearly as the number of passes increased, suggesting that steady improvements in dispersion quality can be obtained by increasing the number of passes through which a preparation is subjected to dispersal. FIG. 3A shows that a three-stack operation format is about 6-7 times more effective as compared to a two-stack operation format.

The operation conditions of the colloid mill (Table 2) were set at 0° gap and a 105 Hz motor speed. FIG. 3B compares the viscosities (measured under steady state shear of 10 s⁻¹) resulting from using multiple passes with either the colloid mill or rotor stator (with a stack of three 8SF generators) to disperse alpha-1,3-glucan. The rotor stator produced dispersions with significantly higher viscosity compared to the colloid mill after both of the first two passes (FIG. 3B). However, after three or more passes with either machine, the viscosity values of dispersion products were similar.

Altogether, these data show that low to medium intensity dispersing units can produce dispersions with viscosities similar to those produced by higher intensity dispersing units (e.g., homogenizer at 8000 psi) by passing a dispersion multiple times through the unit.

### Example 3

### High Viscosity Dispersions of Never-Dried Insoluble Alpha-Glucan Prepared Using High Solids Grinding

Example 1 and 2 show inline techniques that can be used to make high quality dispersions of insoluble alpha-1,3-glucan. However, inline techniques can sometimes be limited by the viscosity of the dispersions themselves, which hampers creating high solids dispersions. For insoluble alpha-1,3-glucan (DPw 800), approximately 10 wt % solids is the upper concentration limit for preparing dispersions using an inline disperser. This upper limit was overcome in this Example by using a batch mode HIGH SHEAR DISPERSER (HSD). HSD is a trade name of a disperser that uses a COWLES blade type disperser and grinds high solid concentration dispersions to reduce particle size.

The insoluble alpha-1,3-glucan used in this Example was identical to the alpha-1,3-glucan used in Example 1. Samples of a 15, 14 and 13 wt % solids wet cake (never-dried) of alpha-1,3-glucan (as prepared in Example 1) were dispersed as described below.

A Hockmeyer Equipment Corp. Model 3-HLI Lab Disperser (an HSD) was used in two stages with the 15 wt % solids wet cake, where initial grinding of alpha-1,3-glucan to a dispersion was performed first at 15 wt % solids for 3 minutes, followed by diluting (or “letting down”) the dispersion to 14 wt % for further grinding (for 2 or 7 more minutes). This let down was necessary to be able to further grind, since the 15 wt % solids preparation provided substantial resistance to grinding. Samples of the 14 and 13 wt % wet cakes were ground with the HSD for 3, 5, or 10 minutes with no let down modification. Two different types of COWLES blades were used, type F or D. While the type F blade requires 42 Hz grinding, the type D blade requires 52 Hz grinding. Each dispersion after grinding was diluted to 8 wt % solids.

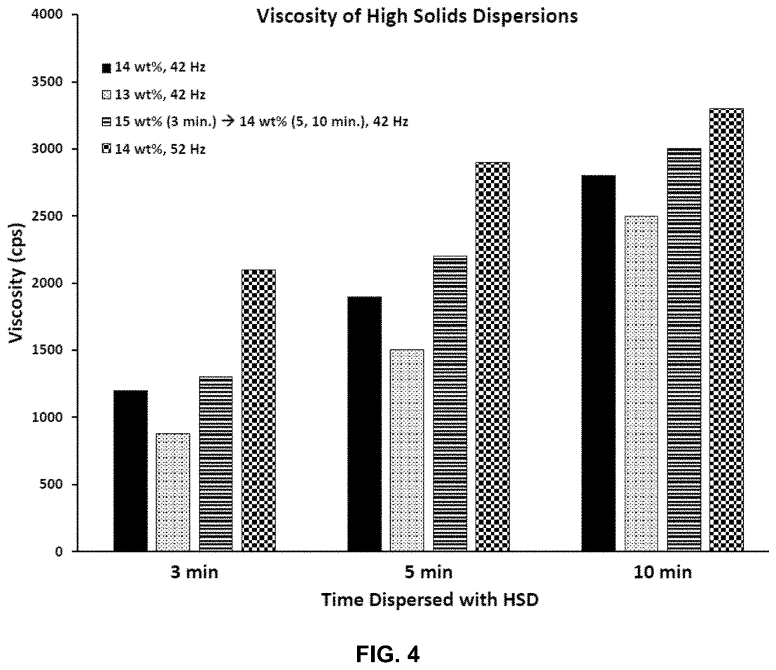

The viscosities of the dispersions were measured at room temperature using a Brookfield DV-II viscometer equipped with an RV-2 spindle (shear of 10 rpm), and are shown in FIG. 4. Overall, the 14 wt % solids dispersions of insoluble alpha-1,3-glucan yielded the highest viscosity measurements (FIG. 4), and providing little difficulty when being grinded by the HSD.

What is claimed is:

1. A method of producing an aqueous dispersion, said method comprising:
(a) providing a first composition comprising at least 88% insoluble alpha-glucan by weight of the first composition, wherein at least 50% of the glycosidic linkages of the insoluble alpha-glucan are alpha-1,3 glycosidic linkages, and
(b) mixing at least aqueous liquid and said first composition to produce an aqueous dispersion having about 0.5% to about 10% by weight of the insoluble alpha-glucan, wherein said mixing comprises applying pressure of at least 1000 pounds per square inch (psi).

2. The method of claim 1, wherein said pressure of at least 1000 psi is applied by pressure homogenization.

3. The method of claim 1, wherein the first composition comprises at least 95% insoluble alpha-glucan by weight of the first composition.

4. The method of claim 1, wherein the aqueous dispersion has about 0.5% to about 4.5% by weight of the insoluble alpha-glucan.

5. The method of claim 1, wherein at least 90% of the glycosidic linkages of the insoluble alpha-glucan are alpha-1,3 linkages.

6. The method of claim 1, wherein the weight average degree of polymerization (DPw) of the insoluble alpha-glucan is at least about 100.

7. The method of claim 1, wherein at least 60% by weight of the insoluble alpha-glucan particles in the aqueous dispersion have a diameter of less than 30 micrometers.

8. The method of claim 1, wherein the insoluble alpha-glucan is dispersed through at least about 60% of the volume of the aqueous dispersion.

9. The method of claim 1, wherein the insoluble alpha-glucan provided in the first composition was dried by agitated air drying.

10. The method of claim 1, wherein the insoluble alpha-glucan was in the form of a wet cake prior to being provided in the first composition, wherein the wet cake comprised about 10% to about 55% by weight of the insoluble alpha-glucan and about 45% to 90% by weight of an aqueous fluid.

11. The method of claim 1, wherein the first composition comprises less than about 0.35% by weight of soluble sugars on a dry weight basis.

12. The method of claim 1, wherein the insoluble alpha-glucan provided in the first composition was produced in an enzymatic reaction comprising at least water, sucrose and a glucosyltransferase enzyme that synthesizes insoluble alpha-glucan at a yield of at least about 75%.

13. The method of claim 1, wherein the insoluble alpha-glucan provided in the first composition was produced in an enzymatic reaction comprising at least water, sucrose, a glucosyltransferase enzyme that synthesizes insoluble alpha-glucan, and oligosaccharides that
(i) comprise alpha-1,3 and alpha-1,6 glycosidic linkages, and/or
(ii) are produced from a glucosyltransferase reaction,
wherein the oligosaccharides were added during preparation of the enzymatic reaction.

14. The method of claim 1, wherein the aqueous dispersion has a viscosity that is at least about 50% higher than the viscosity that the aqueous dispersion would have had if it was instead prepared by mixing at an rpm (revolutions per minute) of no more than 10000.

15. An aqueous dispersion produced according to the method of claim 1.

16. An aqueous dispersion comprising about 0.5 wt % to about 10 wt % insoluble alpha-glucan, wherein:
at least 60% by weight of the insoluble alpha-glucan particles in the aqueous dispersion have a diameter of less than 30 micrometers,
the insoluble alpha-glucan is dispersed through at least about 60% of the volume of the aqueous dispersion, and
at least 50% of the glycosidic linkages of the insoluble alpha-glucan are alpha-1,3 glycosidic linkages.

17. The aqueous dispersion of claim 16, wherein the aqueous dispersion is comprised in a household care product, personal care product, industrial product, pharmaceutical product, or food product.
